SEQUENCE LISTING (34 sequences)

SEQ ID NO: 1 - DNA, 50 bp, Artificial Sequence
  Description of Artificial Sequence Synthetic primer
  modified_base (42)..(42): a, c, t, g, unknown or other
tcgtcggcagcgtcagatgtgtataagagacagcctacgggnggcwgcag

SEQ ID NO: 2 - DNA, 55 bp, Artificial Sequence
  Description of Artificial Sequence Synthetic primer
gtctcgtgggctcggagatgtgtataagagacaggactachvgggtatctaatcc

SEQ ID NO: 3 - DNA, 23 bp, Artificial Sequence
  Description of Artificial Sequence Synthetic primer
ttctctgtaccatgacactctgc

SEQ ID NO: 4 - DNA, 21 bp, Artificial Sequence
  Description of Artificial Sequence Synthetic primer
cgtggaatcttccggctgtag

SEQ ID NO: 5 - DNA, 23 bp, Artificial Sequence
  Description of Artificial Sequence Synthetic primer
ttcctgctgtttctcttacacct

SEQ ID NO: 6 - DNA, 21 bp, Artificial Sequence
  Description of Artificial Sequence Synthetic primer
ctgtctgcctcttttggtcag

SEQ ID NO: 7 - DNA, 20 bp, Artificial Sequence
  Description of Artificial Sequence Synthetic primer
gctgctttgcctacctctcc

SEQ ID NO: 8 - DNA, 21 bp, Artificial Sequence
  Description of Artificial Sequence Synthetic primer
tcgagtgacaaacacgactgc

SEQ ID NO: 9 - DNA, 21 bp, Artificial Sequence
  Description of Artificial Sequence Synthetic primer
tctacgcagtgcttctttgcc

SEQ ID NO: 10 - DNA, 23 bp, Artificial Sequence
  Description of Artificial Sequence Synthetic primer
aagggggatcttcagctttagta

SEQ ID NO: 11 - DNA, 21 bp, Artificial Sequence
  Description of Artificial Sequence Synthetic primer
atgtggacacggaagagacaa

SEQ ID NO: 12 - DNA, 22 bp, Artificial Sequence
  Description of Artificial Sequence Synthetic primer
ggtaagggtaagattggtggtg

SEQ ID NO: 13 - DNA, 23 bp, Artificial Sequence
  Description of Artificial Sequence Synthetic primer
gtgagatacaacgtagctgactg

SEQ ID NO: 14 - DNA, 22 bp, Artificial Sequence
  Description of Artificial Sequence Synthetic primer
tcctgcatccaagatagcaagt

SEQ ID NO: 15 - DNA, 23 bp, Artificial Sequence
  Description of Artificial Sequence Synthetic primer
gctgtgaggataagaacttggag

SEQ ID NO: 16 - DNA, 19 bp, Artificial Sequence
  Description of Artificial Sequence Synthetic primer
ctggttcgacagggctgaa

SEQ ID NO: 17 - DNA, 19 bp, Artificial Sequence
  Description of Artificial Sequence Synthetic primer
cggagtcggaggccctaat

SEQ ID NO: 18 - DNA, 22 bp, Artificial Sequence
  Description of Artificial Sequence Synthetic primer
acagcaggtgcttcttaatgag

SEQ ID NO: 19 - DNA, 20 bp, Artificial Sequence
  Description of Artificial Sequence Synthetic primer
atgaagccgtacttctgccg

SEQ ID NO: 20 - DNA, 22 bp, Artificial Sequence
  Description of Artificial Sequence Synthetic primer
cgcatttttaactgctggacag

SEQ ID NO: 21 - DNA, 23 bp, Artificial Sequence
  Description of Artificial Sequence Synthetic primer
ccctcacactcagatcatcttct

SEQ ID NO: 22 - DNA, 19 bp, Artificial Sequence
  Description of Artificial Sequence Synthetic primer
gctacgacgtgggctacag

SEQ ID NO: 23 - DNA, 23 bp, Artificial Sequence
  Description of Artificial Sequence Synthetic primer
tagtccttcctaccccaatttcc

SEQ ID NO: 24 - DNA, 21 bp, Artificial Sequence
  Description of Artificial Sequence Synthetic primer
ttggtccttagccactccttc

SEQ ID NO: 25 - DNA, 19 bp, Artificial Sequence
  Description of Artificial Sequence Synthetic primer
ccaaccaccaggctacagg

SEQ ID NO: 26 - DNA, 19 bp, Artificial Sequence
  Description of Artificial Sequence Synthetic primer
gcgtcacactcaagctctg

SEQ ID NO: 27 - DNA, 23 bp, Artificial Sequence
  Description of Artificial Sequence Synthetic primer
cacggataaaacgaccataggtg

SEQ ID NO: 28 - DNA, 22 bp, Artificial Sequence
  Description of Artificial Sequence Synthetic primer
tcttgacctgtcattttgccag

SEQ ID NO: 29 - DNA, 20 bp, Artificial Sequence
  Description of Artificial Sequence Synthetic primer
ccactctcgaccctacatgg

SEQ ID NO: 30 - DNA, 22 bp, Artificial Sequence
  Description of Artificial Sequence Synthetic primer
ggcccccaaagtgacatttatt

SEQ ID NO: 31 - DNA, 20 bp, Artificial Sequence
  Description of Artificial Sequence Synthetic primer
gtaacccgttgaaccccatt

SEQ ID NO: 32 - DNA, 20 bp, Artificial Sequence
  Description of Artificial Sequence Synthetic primer
ccatccaatcggtagtagcg

SEQ ID NO: 33 - DNA, 21 bp, Artificial Sequence
  Description of Artificial Sequence Synthetic primer
gatcggtgctaagttctggga

SEQ ID NO: 34 - DNA, 21 bp, Artificial Sequence
  Description of Artificial Sequence Synthetic primer
agggacatacttgccacctgt

(19) United States
(12) Patent Application Publication
(10) Pub. No.: US 2022/0370553 A1
(43) Pub. Date: Nov. 24, 2022

(54) PREBIOTIC-INDUCED ANTI-TUMOR IMMUNITY

(71) Applicant: Sanford Burnham Prebys Medical Discovery Institute, La Jolla, CA (US)

(72) Inventors: Ze'ev A. RONAI, Fallbrook, CA (US); Scott PETERSON, Escondido, CA (US); Yan LI, San Diego, CA (US)

(21) Appl. No.: 17/627,558

(22) PCT Filed: Jul. 30, 2020
(86) PCT No.: PCT/US2020/044309

§ 371 Date: Jan. 14, 2022

Related U.S. Application Data
(60) Provisional application No. 62/880,998, filed on Jul. 31, 2019.

(51) Int. Cl. A61K 38/17; A61K 45/06; A61P 35/00; A61K 31/733

(52) U.S. Cl. CPC A61K 38/1735; A61K 45/06; A61P 35/00; A61K 31/733

(57) ABSTRACT

Described herein are methods and compositions for treating, reducing, or ameliorating cancer in a subject, comprising administering compositions comprising mucin and/or inulin. In some aspects, described herein is a method of enhancing anti-cancer immunity comprising: (a) administering to a subject a composition comprising mucin, wherein the subject has been identified as having a gut microbiome comprising one more microbial taxa that are members of a Clostridium cluster XIVa or an Actinobacteria phylum; and (b) altering the gut microbiome in the subject, wherein administration of the composition causes an enhanced anti-cancer immunity in the subject.

### CROSS-REFERENCE TO RELATED APPLICATIONS

This application claims the benefit of U.S. Provisional Patent Application No. 62/880,998 filed on Jul. 31, 2019. Priority is claimed pursuant to 35 U.S.C. § 119. The above noted patent application is incorporated by reference as if set forth fully herein.

### STATEMENT REGARDING FEDERALLY SPONSORED RESEARCH

This invention was made with government support under R01 CA216187 and R35 CA197465 awarded by the National Institutes of Health (NIH), W81XWH-16-1-0517 from the Department of Defense, and grant 509524 awarded by the Melanoma Research Alliance. The government has certain rights in the invention.

### FIELD

The compositions and methods described herein relate generally to the fields of prebiotics, cancer, and anti-cancer immunity.

### BACKGROUND

The gastrointestinal (GI) tract harbors a complex and dynamic population of bacteria referred to as the gut microbiota. The gut microbiota can affect key components of host physiology and homeostasis, and the composition of the gut microbiota is implicated in the maintenance of health in the onset and progression of disease, including cancer. Prebiotics can alter the composition of the microbiota, for example, by providing nutrients that favor expansion of certain microbial taxa.

### INCORPORATION BY REFERENCE

Each patent, publication, and non-patent literature cited in the application is hereby incorporated by reference for such disclosure as if each was incorporated by reference individually.

### SUMMARY

In some aspects, described herein is a method of enhancing anti-cancer immunity comprising: (a) administering to a subject a composition comprising mucin, wherein the subject has been identified as having a gut microbiome comprising one more microbial taxa that are members of a Clostridium cluster XIVa or an Actinobacteria phylum; and (b) altering the gut microbiome in the subject, wherein administration of the composition causes an enhanced anti-cancer immunity in the subject.

In some embodiments, the altering the gut microbiome comprises increasing an abundance of the one or more microbial taxa by at least 10%. In some embodiments, the altering the gut microbiome comprises increasing an abundance of a microbial population by at least 10%. In some embodiments, the microbial population is selected from the group consisting of: a microbial population that promotes inflammation, a microbial population that reduces inflammation, and a microbial population that is negatively correlated with tumor progression. In some embodiments, the altering the gut microbiome comprises reducing an abundance of a microbial population by at least 10%. In some embodiments, the microbial population is selected from the group consisting of: a microbial population that promotes inflammation, a microbial population that reduces inflammation, and a microbial population that is positively correlated with tumor progression. In some embodiments, the altering the gut microbiome comprises increasing an abundance of a taxonomic unit by at least 10%. In some embodiments, the taxonomic unit comprises a species selected from the group consisting of: a Clostriales species, a Bacteroides species, a Barnesiella species, a Parasutterella species, a Bifidobacterium species, an Olsenella species, a Parabacteroides species, a Dorea species, a Lachnospiraceae species, an Acetatifactor species, a Robinsoniella species, a Mobilitalea species, a Eubacterium species, an Eisenbergiella species, a Lachnotalea species, a Prevotellamassilia species, a Culturomica species, a Firmicutes species, a Pseudoflavonifractor species, a Tyzzerella species, an Anaerostipes species, a Proteobacteria species, a Halovibrio species, a Tenericutes species, and a Chlorflexi species. In some embodiments, altering the gut microbiome comprises increasing a diversity of glycosyl hydrolases encoded by the gut microbiome by at least 10%. In some embodiments, altering the gut microbiome comprises increasing a diversity of glycosyl hydrolases expressed by the gut microbiome by at least 10%. In some embodiments, the method reduces tumor growth in the subject by at least 10%. In some embodiments, the method reduces cancer progression in the subject. In some embodiments, the cancer is a skin cancer. In some embodiments, the cancer is a colorectal cancer. In some embodiments, the method further comprises administering to the subject an anti-cancer therapy. In some embodiments, the anti-cancer therapy is selected from the group consisting of: radiotherapy, chemotherapy, immunotherapy, a chemical compound, a small molecule, a kinase inhibitor, a checkpoint inhibitor, and a cellular therapy. In some embodiments, administering the anti-cancer therapy and the composition comprising mucin modifies the gut microbiome of the subject relative to administering only the composition comprising mucin. In some embodiments, administering the anti-cancer therapy and the composition comprising mucin increases an abundance of a taxonomic unit by at least 10% relative to administering to the subject a composition comprising mucin. In some embodiments, the taxonomic unit is selected from the group consisting of: an Akkermansia species, an Actinobacteria species, a Bizdobacterium species, an Olsenella species, and a Parvibacter species. In some embodiments, the enhanced anti-cancer immunity is characterized by a stimulated anti-tumor immune response. In some embodiments, the enhanced anti-cancer immunity is characterized by a stimulated pro-inflammatory immune response in a tumor microenvironment. In some embodiments, the enhanced anti-cancer immunity comprises an increased tumor infiltration of at least 10% by cells selected from the group consisting of: CD4+ T cells, CD8+ T cells, CD45+ cells, dendritic cells, plasmacytoid dendritic cells, and CD8a+ dendritic cells. In some embodiments, the enhanced anti-cancer immunity comprises an increased intra-tumoral expression of at least 10% of a gene selected from the group consisting of: an immune system gene, a cytokine gene, a chemokine gene, a gene involved in antigen presentation, a MHC-I gene, and a MHC-II gene. In some embodiments, the method increases a concentration of a cytokine or chemokine in the subject's blood by at least 10%. In some embodiments, the method decreases a concentration of a cytokine or chemokine in the subject's blood by at least 10%. In some embodiments, the method increases expression of CD40, CD80, MHC-I, or MHC-II by dendritic cells in the subject by at least 10%. In some embodiments, the method increases T cell activation in the subject by at least 10%. In some embodiments, the method increases T cell expression of a cytokine, chemokine, or granzyme B in the subject by at least 10%. In some embodiments, the method increases expression of an immune-related gene by intestinal epithelial cells in the subject by at least 10%. In some embodiments, the method increases expression of a cytokine or chemokine by intestinal epithelial cells in the subject by at least 10%.

In some aspects, described herein is a method of enhancing anti-cancer immunity comprising: (a) administering to a subject a composition comprising inulin, wherein the subject has been identified as having a gut microbiome comprising one more microbial taxa that are members of a Clostridium cluster XIVa or an Actinobacteria phylum; and (b) altering the gut microbiome in the subject, wherein administration of the composition causes an enhanced anti-cancer immunity in the subject.

In some embodiments, the altering the gut microbiome comprises increasing an abundance of the one or more microbial taxa by at least 10%. In some embodiments, the altering the gut microbiome comprises increasing an abundance of a microbial population by at least 10%. In some embodiments, the microbial population is selected from the group consisting of: a microbial population that promotes inflammation, a microbial population that reduces inflammation, and a microbial population that is negatively correlated with tumor progression. In some embodiments, the altering the gut microbiome comprises reducing an abundance of a microbial population by at least 10%. In some embodiments, the microbial population is selected from the group consisting of: a microbial population that promotes inflammation, a microbial population that reduces inflammation, and a microbial population that is positively correlated with tumor progression. In some embodiments, the altering the gut microbiome comprises increasing an abundance of a taxonomic unit by at least 10%. In some embodiments, the taxonomic unit comprises a species selected from the group consisting of: a Clostriales species, a Bacteroides species, a Barnesiella species, a Parasutterella species, a Bifidobacterium species, an Olsenella species, a Parabacteroides species, a Dorea species, a Lachnospiraceae species, an Acetatifactor species, a Robinsoniella species, a Mobilitalea species, a Eubacterium species, an Eisenbergiella species, a Lachnotalea species, a Prevotellamassilia species, a Culturomica species, a Firmicutes species, a Pseudoflavonifractor species, a Tyzzerella species, an Anaerostipes species, a Proteobacteria species, a Halovibrio species, a Tenericutes species, and a Chlorflexi species. In some embodiments, altering the gut microbiome comprises increasing a diversity of glycosyl hydrolases encoded by the gut microbiome by at least 10%. In some embodiments, altering the gut microbiome comprises increasing a diversity of glycosyl hydrolases expressed by the gut microbiome by at least 10%. In some embodiments, the method reduces tumor growth in the subject by at least 10%. In some embodiments, the method reduces cancer progression in the subject. In some embodiments, the cancer is a skin cancer. In some embodiments, the cancer is a colorectal cancer. In some embodiments, the method further comprises administering to the subject an anti-cancer therapy. In some embodiments, the anti-cancer therapy is selected from the group consisting of: radiotherapy, chemotherapy, immunotherapy, a chemical compound, a small molecule, a kinase inhibitor, a checkpoint inhibitor, and a cellular therapy. In some embodiments, the anti-cancer therapy and the composition comprising inulin modifies the gut microbiome of the subject relative to administering only the composition comprising inulin. In some embodiments, the anti-cancer therapy and the composition comprising inulin increases an abundance of a taxonomic unit by at least 10% relative to administering to the subject a composition comprising inulin. In some embodiments, the taxonomic unit is selected from the group consisting of: an Akkermansia species, an Actinobacteria species, a Bifidobacterium species, an Olsenella species, and a Parvibacter species. In some embodiments, the enhanced anti-cancer immunity is characterized by a stimulated anti-tumor immune response. In some embodiments, the enhanced anti-cancer immunity is characterized by a stimulated pro-inflammatory immune response in a tumor microenvironment. In some embodiments, the enhanced anti-cancer immunity comprises an increased tumor infiltration of at least 10% by cells selected from the group consisting of: CD4+ T cells, CD8+ T cells, CD45+ cells, dendritic cells, plasmacytoid dendritic cells, and CD8a+ dendritic cells. In some embodiments, the enhanced anti-cancer immunity comprises an increased intra-tumoral expression of at least 10% of a gene selected from the group consisting of: an immune system gene, a cytokine gene, a chemokine gene, a gene involved in antigen presentation, a MHC-I gene, and a MHC-II gene. In some embodiments, the method increases a concentration of a cytokine or chemokine in the subject's blood by at least 10%. In some embodiments, the method decreases a concentration of a cytokine or chemokine in the subject's blood by at least 10%. In some embodiments, the method increases expression of CD40, CD80, MHC-I, or MHC-II by dendritic cells in the subject by at least 10%. In some embodiments, the method increases T cell activation in the subject by at least 10%. In some embodiments, the method increases T cell expression of a cytokine, chemokine, or granzyme B in the subject by at least 10%. In some embodiments, the method increases expression of an immune-related gene by intestinal epithelial cells in the subject by at least 10%. In some embodiments, the method increases expression of a cytokine or chemokine by intestinal epithelial cells in the subject by at least 10%.

### BRIEF DESCRIPTION OF THE FIGURES

The patent application contains at least one drawing executed in color. Copies of this patent or patent application with color drawings will be provided by the Office upon request and payment of the necessary fee.

The novel features of the disclosed methods are set forth with particularity in the appended claims. A better understanding of the features and advantages of the disclosed methods will be obtained by reference to the following detailed description that sets forth illustrative embodiments, in which the principles of the disclosed methods are utilized, and the accompany drawings of which:

FIG. 1 shows that prebiotics enrich for anti-tumor promoting taxa in vitro. Fecal samples derived from 12 healthy human subjects were cultivated in the presence or absence of 1% prebiotic. 16S rDNA sequences corresponding to Bifidobacterium, Bacteroides and Akkermansia muciniphila were quantified. Data are representative of three independent experiments. Graphs show the mean±s.e.m. *P<0.05, **P<0.005, ***P<0.001, ****P<0.0001 by one-way ANOVA with Tukey's correction.

FIG. 2 demonstrates that administration of mucin or inulin reduces tumor growth and induces anti-tumor immunity. FIG. 2A, Growth of Yumm1.5 tumors in C57BL/6 mice provided with 0 or 3% mucin in drinking water or received a control diet or a diet enriched 15% inulin 14 days prior to and during tumor inoculation (n=15). FIG. 2B, Quantification of tumor-infiltrating effector (CD44hi) CD4+, CD8+ and CD45+ T cells from mice treated as in FIG. 2A (n=10). FIG. 2C, Quantification of tumor-infiltrating, IFN-g-producing CD4+ T cells from mice treated as in FIG. 2A (n=10). FIG. 2D, Quantification of tumor-infiltrating total DCs and subsets in mice treated as in FIG. 2A (n=10). FIG. 2E, MFI of MHC class I and MHC class II on DCs from the tumors of mice treated as in FIG. 2A (n=10). Data are representative of three independent experiments. Graphs show the mean s.e.m. *P<0.05, **P<0.005, ***P<0.001,****P<0.0001 by two-way ANOVA with Tukey's correction (A) and by one-way ANOVA with Tukey's correction (B-E).

FIG. 3 illustrates qPCR of immune-related gene expression in tumors isolated from C57BL/6 mice that received either a control diet alone, or one supplemented with mucin (3%) in drinking water or with inulin (15%) in chow starting 14 days prior to tumor inoculation (n=6). Data are representative of three independent experiments. Graphs show the mean±s.e.m. *P<0.05, **P<0.005, ***P<0.001, and by one-way ANOVA with Tukey's correction.

FIG. 4 demonstrates that mucin increases the frequency and number of tumor-specific CD8+ T cells in tumor-draining lymph nodes. FIG. 4A Quantification of CD45.1+OT-I CD8+ T cell frequencies in the tumor-draining lymph nodes (TdLN) and non-draining lymph nodes (ndLN) of C57BL/6 mice (CD45.2+) treated with or without mucin and injected with B16-OVA melanoma cells (n=6). Right dot plots show gating of CD45.1+CD8+ cells (n=6). FIG. 4B Quantification of CD45.1+OT-I CD8+ T cell number in the tumor-draining lymph nodes (TdLN) and non-draining lymph nodes (ndLN) of WT C57BL/6 mice CD45.2+ mice that were injected with B16-OVA melanoma cells (n=10). Graphs show the mean s.e.m. *P<0.05, by two-tailed t-test or Mann-Whitney U test.

FIG. 5 demonstrates the effect of prebiotic treatment on serum cytokine and chemokine levels. FIG. 5A Serum cytokines and chemokines in naïve WT mice treated with or without mucin, without tumor inoculation (n=10). FIG. 5B Serum cytokines in WT mice treated with or without mucin 10 days after tumor inoculation (n=10). FIG. 5C Serum chemokines in WT mice with or without mucin treatment at 10 days after tumor inoculation (n=10). Graphs show the mean±s.e.m. *P<0.05, ****P<0.0001 by two-tailed t-test or Mann-Whitney U test.

FIG. 6 demonstrates alterations in the composition and diversity of gut microbiota from mucin or inulin treatment. FIG. 6A, Principal Component Analysis (PCA) of all taxa enumerated in mucin treated and control mice fecal microbiota samples taken at different time points (A, before mucin treatment; B, before tumor injection; C, before tumor collection) after injection of YUMM1.5 tumor cells. FIG. 6B, PCA of all taxa enumerated in inulin treated and control mice fecal microbiota samples taken at different time points (A, before inulin treatment; B, before tumor injection; C, before tumor collection) after injection of YUMM1.5 tumor cells. FIG. 6C, Boxplot of the relative abundance of the 6 taxa enriched in inulin-treated mice microbiota that are negatively correlated with tumor size (control, n=12; inulin, n=15; mucin, n=15). *P<0.05, **P<0.005, ***P<0.001, ****P<0.00011 and by one-way ANOVA with Tukey's correction.

FIG. 7 Illustrates that MEK inhibitor resistance in a melanoma model can be overcome via combination with inulin. FIG. 7A C57BL/6 mice were injected (s.c.) with NRAS^{Q61K} mouse melanoma cells (1×10⁶) (n=10). The mice were administered with either a control diet alone, or one supplemented with mucin (3%) in drinking water or inulin (15%) in chow starting 14 days prior to tumor inoculation. When tumors reached a volume of 10-20 mm², mice were treated with MEKi (PD325901) administered by gavage (10 mg/kg, daily), alone or in combination with inulin or mucin, as indicated. Tumor volume was assessed every 4 days. FIG. 7B Number of tumor-infiltrating effector (CD44hi) CD4+ and CD8+ T cells, and CD45+ cells per tumor weight (g) from mice treated as described for FIG. 7A. (n=8). FIG. 7C Number of tumor-infiltrating DCs and DC subsets numbers per tumor weight (g) and expression of MHC I on DCs from mice treated as described for FIG. 7A (n=8). FIG. 7D Quantification of NRAS^{Q61K} tumor-infiltrating IFN-g-producing CD4+ and CD8+ T cells from C57BL/6 mice treated with MEKi+mucin or inulin (n=8). Data are representative of two independent experiments. Graphs show the mean±s.e.m. *P<0.05, **P<0.005, ***P<0.001, ****P<0.0001 by two-way ANOVA with Tukey's correction (A) or one-way ANOVA with Tukey's correction (B-D).

FIG. 8 provides prebiotic-induced alterations in microbiota associated with control of N-Ras melanoma tumors and overcoming MEKi inhibitor resistance. FIG. 8A, Pie chart of taxa enriched in inulin treated-mice microbiota that are negatively correlated with MaN-Ras tumor size (n=10). FIG. 8B, Pie chart of taxa enriched in mucin-treated mice microbiota that are negatively correlated with MaN-Ras tumor size (n=10). FIG. 8C, Relative abundance of taxa enriched in mice subjected to treatment of inulin with MEKi, that are negatively correlated with MaN-Ras tumor size (n=10). FIG. 8D, Relative abundance of taxa enriched in mice subjected to treatment with mucin and MEKi, that are negatively correlated with MaN-Ras tumor size (n=10). Data are representative of two independent experiments.

FIG. 9 illustrates changes in the relative abundance of taxa in inulin or mucin-treated mice that are negatively correlated with tumor size. FIG. 9A, Boxplot of the relative abundance of the taxa enriched in inulin treated-mice gut microbiota that are negatively correlated with tumor size (n=10). The fecal samples were taken at different time points (A, before inulin treatment; B, before tumor injection; C, before MEKi treatment, D, before tumor collection) after injection of YUMM1.5 tumor cells. FIG. 9B, Boxplot of the relative abundance of the taxa enriched in gut microbiota of mucin treated-mice, which are negatively correlated with tumor size (n=10). The fecal samples were taken at different time points (A, before mucin treatment; B, before tumor injection; C, before MEKi treatment, D, before tumor collection) following the inoculation of YUMM1.5 tumor cells.

FIG. 10 demonstrates that inulin attenuates colon cancer growth. FIG. 10A, Growth of MC-38 mouse colorectal cancer cells inoculated (1×10⁶) in C57BL/6 mice that were fed with 0 or 3% mucin in drinking water or a diet enriched with 15% inulin 14 days prior to and during tumor inoculation (n=12). FIG. 10B, MFI of MHC II and MHC I in MC-38 tumor-infiltrating DCs of mucin or inulin-treated C57BL/6 mice (n=8). FIG. 10C, Quantification of MC-38 tumor-infiltrating effector (CD44hi) CD4+ and CD8+ T cells and CD45+ cells in mucin or inulin-treated C57BL/6 mice (n=8); FIG. 10D, Quantification of MC-38 tumor-infiltrating IFN-g-producing CD4+ and CD8+ T cells in mucin or inulin-treated C57BL/6 mice (n=8); FIG. 10E, Quantification of MC-38 tumor-infiltrating total DCs and DC subsets in mucin or inulin-treated C57BL/6 mice (n=8).

FIG. 11 illustrates changes in the relative abundance of taxa in inulin-treated mice that are negatively correlated with tumor size in a colorectal cancer model. C57BL/6 mice were fed with 0 or 3% mucin in drinking water or a diet enriched 15% inulin 14 days prior to inoculation with 1×10⁶ MC-38 colorectal cancer cells. A boxplot is provided of the relative abundance of the taxa enriched in inulin treated-mice microbiota that are positively correlated with tumor size (n=10). Data are representative of two independent experiments.

FIG. 12 provides a cladogram representation of taxa enriched in mucin-fed mice microbiota (red) and taxa enriched in inulin fed mice microbiota (blue).

FIG. 13 demonstrates that mucin induced tumor control is dependent on gut microbiota. Germ free C3H/HeN mice were colonized with a minimal microbiota (ASF) to induce immune maturation for two weeks, followed by two weeks of mucin treatment via oral gavage, after which SW1 tumor cells were inoculated. Tumor size was monitored over the next 24 days (n=15).

FIG. 14 illustrates effects of mucin and inulin on the activation of dendritic cells and T cells in vitro. FIG. 14A, MHC I, MHC II, CD40, and CD86 expression (MFI) on BMDCs left untreated (control) or stimulated with 0.05 mg/ml and 0.5 mg/ml mucin and inulin in vitro (n=4). FIG. 14B, qRT-PCR analysis of the indicated cytokine and chemokine mRNAs in CD8+ T cells left untreated (control) or stimulated with 0.05 mg/ml or 0.5 mg/ml mucin or inulin in vitro (n=4). Data are representative of two independent experiments. Graphs show the mean±s.e.m. *P<0.05, **P<0.005, ***P<0.001, ****P<0.0001 one-way ANOVA with Tukey's correction.

FIG. 15 illustrates effects of mucin and inulin on expression of inflammatory mediators by intestinal epithelial cells in vivo. qRT-PCR data are shown for mRNA levels of the indicated inflammatory mediators in intestinal epithelial cells from mucin or inulin-treated mice (n=4). Data are representative of two independent experiments. Graphs show the mean s.e.m. *P<0.05, **P<0.005, one-way ANOVA with Tukey's correction.

FIG. 16 shows that prebiotic therapy exhibits comparable efficacy as anti-PD-1 immune checkpoint therapy. FIG. 16A, Growth of Yumm1.5 tumors in C57BL/6 mice that were subjected to a control diet or a diet enriched with 15% inulin 14 days prior to (and during) tumor inoculation (n=10). The mice were injected with control IgG or with anti-PD-1 blocking antibody (GoInVivo; BioLegend) on days 7, 10, 13, and 16, after tumor inoculation (n=10). FIG. 16B, Growth of Yumm1.5 tumors in C57BL/6 mice administered with 0 or 3% mucin in drinking water for 14 days prior to tumor inoculation (n=10). The mice injected with antibodies noted in A (n=10). Data are representative of two independent experiments. Graphs depict the mean±s.e.m. *P<0.05, ***P<0.001, by two-way ANOVA with Tukey's correction.

FIG. 17 illustrates tumor growth inhibition by combination of mucin and inulin. FIG. 17A Growth of SW1 mouse melanoma cells in C3H/HeOuJ mice that were fed with 0 or 3% mucin in drinking water and/or a diet enriched with 15% inulin, starting 14 days prior to (and continued after) tumor inoculation (n=10). FIG. 17B. Growth of Yumm1.5 mouse melanoma cells in C57BL/6 mice that were fed with 0 or 3% mucin in drinking water and (or) a diet enriched 15% inulin, starting 14 days prior to and during tumor inoculation (n=10). Graphs show the mean±s.e.m. *P<0.05, **P<0.005,***P<0.001, ****P<0.0001 by two-way ANOVA with Tukey's correction.

### DETAILED DESCRIPTION

### Overview

The gastrointestinal (GI) tract harbors a complex and dynamic population of bacteria referred to as the gut microbiota. The gut microbiota can affect key components of host physiology and homeostasis, and the composition of the gut microbiota is implicated in the maintenance of health in the onset and progression of disease, including cancer. Alterations in gut microbiota composition have been associated with, for example, the development and function of the immune system, cancer progression or control, and responsiveness to anti-cancer therapies. Strategies that alter the gut microbiota have the potential to reduce cancer growth or progression, for example, by promoting more effective anti-cancer immune responses.

Prebiotics can alter the composition of the microbiota, for example, by providing nutrients that favor expansion of certain microbial taxa. Provided herein, in some embodiments, are methods for treating, reducing, or ameliorating cancer in a subject, by administering to the subject at least one prebiotic. For example, mucin and inulin are shown to promote expansion of microbial taxa that negatively correlate with tumor size, and promote anti-tumor immune responses in the subject.

In some embodiments, administering a prebiotic of the disclosure to a subject enhances an immune response in a subject. For example, administering mucin or inulin can result in alterations in the microbiota that potentiate or enhance an anti-tumor immune response. In some embodiments, administering a prebiotic of the disclosure increases infiltration of a subset of immune cells into a tumor, alters expression of an immune system-related gene, or a combination thereof.

### Prebiotics

Prebiotics can alter the composition of the microbiota (e.g., the gut microbiota). A prebiotic can be a substrate that is selectively utilized by a certain microorganism, for example, a microorganism that confers a health benefit to a host. A prebiotic can be, for example, metabolizable by microbial enzymes and non-metabolizable by human enzymes.

Administering a prebiotic to a subject can alter the composition of a microbiota, for example, promoting expansion of one or more microbial populations associated with a health benefit. In some embodiments, a prebiotic selectively stimulates the growth and/or activity of one or a limited number of microbial taxa in the digestive tract. In some embodiments, administering a prebiotic as disclosed herein can alter the gut microbiota of a subject to promote anti-tumor immunity in the subject.

A prebiotic can be administered as a component of a food. For example, a prebiotic can naturally occur in a plant, or can be added to a food product to be consumed by a subject (e.g., a yogurt, cereal, bread, biscuit, cookie, dessert, or drink). A prebiotic can be administered as part of a prebiotic composition, as part of a pharmaceutical composition, in a unit dosage form, or a combination thereof. A prebiotic can be administered to a subject at any dose required to produce a desired effect on a microbiota.

A prebiotic can be a carbohydrate or a non-carbohydrate substance. A prebiotic can be a soluble fiber. Examples of prebiotics include, but are not limited to, mucin, inulin, oligosaccharides, galacto-oligosaccharides (GOS), fructo-oligosaccharides (FOS), mannan-oligosaccharide (MOS), Xylooligosaccharides (XOS), human milk oligosaccharides (HMO) oligofructose (OF), chicory fibre, conjugated linoleic acids (CLA), polydextrose, polydextrose powder, lactulose, lactosucrose, raffinose, gluco-oligosaccharide, isomalto-oligosaccharides, soybean oligosaccharides, lactosucrose chito-oligosaccharide, aribino-oligosaccharide, siallyl-oligosaccharide, fuco-oligosaccharide, gentio-oligosaccharides polyunsaturated fatty acids (PUFA), phenolics, and phytochemicals.

In some embodiments, a prebiotic of the disclosure is inulin. Inulin belongs to the fructan carbohydrate subgroup. Depending on its chain length, inulin can be classified as either an oligo- or polysaccharide. It is comprised of β-d-fructosyl subgroups linked together by (2-1) glycosidic bonds and the molecule usually ends with a (1↔2) bonded α-d-glucosyl group. The length of these fructose chains varies and ranges from 2 to 60 monomers. Inulin containing maximally 10 fructose units is also referred to as oligofructose. Inulin is a unique oligo- or polysaccharide because its backbone does not incorporate any sugar ring. Furthermore, inulin is built up mostly from furanose groups, which are more flexible than pyranose rings. This translates into a greater freedom to move and thus more molecular flexibility of the molecule compared to other oligo- and polysaccharides, because of its (2-1) linked-d-fructosyl backbone. Inulin has a higher molecular weight than mono- and di-saccharides, the higher molecular weight also correlates with a lower solubility.

Inulin can be found in a wide range of plants, including fruits, vegetables, and herbs, including wheat, onions, bananas, leeks, artichokes, asparagus, and chicory roots. Most commercially available inulin is extracted from chicory root, which contains a relatively high concentration of this carbohydrate. Apart from extraction from plants, inulin can also be produced enzymatically. As a food additive, oligofructose can be used a sweet-replacer and longer chain inulin can be used as a fat replacer and texture modifier. Both inulin and oligofructose can be used as dietary fiber and prebiotics in functional foods.

Inulin is not metabolized by human metabolic enzymes, but can be utilized by certain microbes within the gut microbiota. In some embodiments, administering inulin to a subject can alter the gut microbiota of the subject to promote anti-tumor immunity.

In some embodiments, a prebiotic of the disclosure is mucin. Mucins are a family of high molecular weight, heavily glycosylated proteins (glycoconjugates) produced by epithelial tissues in most metazoans. Examples of genes encoding mucin proteins include, but are not limited to, MUC2, MUC5AC, MUC5B, MUC6, MUC7, MUC9, MUC19, MUC1, MUC3A/B, MUC4, MUC12, MUC13, MUC15, MUC16, MUC17, MUC20, and MUC21. Mucin genes encode mucin monomers that are synthesized as rod-shaped apomucin cores that are post-translationally modified by abundant glycosylation. Two distinctly different regions are found in mature mucins: (i) the amino- and carboxy-terminal regions are lightly glycosylated, but rich in cysteines, which are likely involved in establishing disulfide linkages within and among mucin monomers; and (ii) a large central region formed of multiple tandem repeats of 10 to 80 residue sequences, in which up to half of the amino acids are serine or threonine. This area becomes saturated with O-linked and N-linked oligosaccharides. The O-glycan structures present in mucin are diverse and complex, consisting predominantly of core 1-4 mucin-type O-glycans containing α- and β-linked N-acetyl-galactosamine, galactose and N-acetyl-glucosamine. These core structures are further elongated and frequently modified by fucose and sialic acid sugar residues via α1,2/3/4 and α2,3/6 linkages, respectively. The dense “sugar coating” of mucins gives them considerable water-holding capacity and also makes them resistant to proteolysis. Mucins are secreted as aggregates with molecular masses. Within these aggregates, monomers are linked to one another mostly by non-covalent interactions, although intermolecular disulfide bonds may also play a role in this process. Mucins can form a gel-like layer on the surface of the gut epithelium which can act as lubrication and a protective barrier.

Mucins can be utilized by certain microbes within the gut microbiota. For example, the ability to metabolize mucin or mucin O-linked oligosaccharides may contribute to the ability of a microbe to colonize the mucosal surface. Due to their proximity to the immune system, mucin-degrading bacteria may be in a prime location to influence the host response. In some embodiments, administering mucin to a subject can alter the gut microbiota of the subject to promote anti-tumor immunity.

In some embodiments, a subject is administered two or more prebiotics, e.g., administered mucin and inulin.

### Alterations to Microbiota

The gastrointestinal (GI) tract harbors a complex and dynamic population of bacteria referred to as the gut microbiota. The gut microbiota can affect key components of host physiology and homeostasis, and the composition of the gut microbiota is implicated in the maintenance of health in the onset and progression of diseases, including cancer. Alterations in gut microbiota composition have been associated with, for example, the development and function of the immune system, cancer progression or control, and responsiveness to anti-cancer therapies. Strategies that alter the gut microbiota have the potential to reduce cancer growth or progression, for example, by promoting more effective anti-cancer immune responses.

In some embodiments, administering a prebiotic to a subject as disclosed herein results in alteration of the subject's gut microbiota. Alterations to the microbiota can comprise increasing or decreasing the concentration of a microbial taxonomic unit in the gut microbiota (e.g., the concentration per gram in gut luminal contents or feces). Alterations to the gut microbiota can comprise increasing or decreasing the relative concentration of a microbial taxonomic unit in the gut microbiota (e.g., the percentage or relative proportion of a taxonomic unit within the total gut microbiota).

In some embodiments, administering a prebiotic of the disclosure to a subject can increase the abundance of a taxonomic unit by at least 10%, 20%, 30%, 40%, 50%, 60%, 70%, 80%, 90%, 2.0-fold, 2.1-fold, 2.2-fold, 2.3-fold, 2.4-fold, 2.5-fold, 2.6-fold, 2.7-fold, 2.8-fold, 3.0-fold, 3.1-fold, 3.2-fold, 3.3-fold, 3.4-fold, 3.5-fold, 4-fold, 5-fold, 6-fold, 7-fold, 8-fold, 9-fold, 10-fold, 15-fold, 20-fold, 30-fold, 40-fold, 50-fold, 100-fold or more in a subject.

In some embodiments, administering a prebiotic of the disclosure to a subject can decrease the abundance of a taxonomic unit by at least 10%, 20%, 30%, 40%, 50%, 60%, 70%, 80%, 90%, 2.0-fold, 2.1-fold, 2.2-fold, 2.3-fold, 2.4-fold, 2.5-fold, 2.6-fold, 2.7-fold, 2.8-fold, 3.0-fold, 3.1-fold, 3.2-fold, 3.3-fold, 3.4-fold, 3.5-fold, 4-fold, 5-fold, 6-fold, 7-fold, 8-fold, 9-fold, 10-fold, 15-fold, 20-fold, 30-fold, 40-fold, 50-fold, 100-fold or more in a subject.

In some embodiments, methods of the disclosure comprise identifying and/or quantifying a microbial taxonomic unit in the gut microbiota. In some embodiments, methods of the disclosure comprise determining whether a microbial taxonomic unit is present in the gut microbiota. In some embodiments, methods of the disclosure comprise quantifying the absolute or relative abundance of a microbial taxonomic unit in the gut microbiota. The presence or abundance of a microbial taxonomic unit can be determined, for example, by processing a biological sample obtained from a subject (e.g., a fecal sample or a biopsy sample). In some embodiments, nucleic acids can be extracted from a biological sample and processed for sequencing. In some embodiments, nucleic acids are enriched for sequences of interest prior to sequencing, for example, enriched for ribosomal RNA sequences using PCR with suitable primers.

The biological samples can be obtained from a subject at different stages of disease progression. Different stages of disease progression or can include healthy, at the onset of primary symptom, at the onset of secondary symptom, at the onset of tertiary symptom, during the course of primary symptom, during the course of secondary symptom, during the course of tertiary symptom, at the end of the primary symptom, at the end of the secondary symptom, at the end of tertiary symptom, after the end of the primary symptom, after the end of the secondary symptom, after the end of the tertiary symptom, or a combination thereof. Different stages of disease progression can be a period of time after being diagnosed or suspected to have a disease; for example, about, or at least, 1, 2, 3, 4, 5, 6, 7, 8, 9, 10, 11, 12, 13, 14, 15, 16, 17, 18, 19, 20, 21, 22, 23 or 24 hours; 1, 2, 3, 4, 5, 6, 7, 8, 9, 10, 11, 12, 13, 14, 15, 16, 17, 18, 19, 20, 21, 22, 23, 24, 25, 26, 27 or 28 days; 1, 2, 3, 4, 5, 6, 7, 8, 9, 10, 11, 12, 13, 14, 15, 16, 17, 18, 19 or 20 weeks; 1, 2, 3, 4, 5, 6, 7, 8, 9, 10, 11 or 12 months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 years after being diagnosed or suspected to have a disease. Different stages of disease progression or different conditions can include before, during or after an action or state; for example, treatment with drugs, treatment with a surgery, treatment with a procedure, performance of a standard of care procedure, resting, sleeping, eating, fasting, and the like.

Taxonomic units in the gut microbiota can be identified and/or quantified at various levels, for example, at kingdom, phylum, class, order, family, genus, species, subspecies, strain, or substrain level, or a combination thereof. Taxonomic units in the gut microbiota can be identified and/or quantified as phylotypes or Operational Taxonomic Units (OTUs, e.g., a group of sequences sharing at least a specified level of similarity to a particular nucleic acid sequence).

In some embodiments, a taxonomic unit can be identified and/or quantified by sequencing a nucleic acid sequence. A taxonomic unit can be identified and/or quantified by sequencing, for example, a sequence encoding part or all of a ribosomal RNA (rRNA, e.g., a 16S rRNA, a 23s rRNA, an 18S rRNA, a 28S rRNA, a 5S rRNA, a 5.8S rRNA, or a combination thereof). In some embodiments, a taxonomic unit is identified and/or quantified by sequencing a one or more hypervariable regions of a 16S rRNA sequence (e.g., a V1, V2, V3, V4, V5, V6, V7, V8, V9, or a combination thereof).

Members of a taxonomic unit can share, for example, at least 50%, 55%, 60%, 65%, 70%, 75%, 80%, 85%, 86%, 87%, 88%, 89%, 90%, 91%, 92%, 93%, 94%, 95%, 95.5% 96%, 96.5%, 97%, 97.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99%, or more sequence identity of a particular nucleic acid sequence (e.g., a ribosomal RNA sequence).

In some embodiments, administering a prebiotic of the disclosure to a subject can alter the abundance of a taxonomic unit (e.g., phylum, class, order, family, genus, species, subspecies, strain, substrain, phylotype, or OTU) associated with or comprising any one or more of Acetatifactor, Acetatifactor muris, Acholeplasma, Acholeplasma pleciae, Acholeplasmataceae, Actinobacteria, Akkermansia, Akkermansia muniniphila, Alistipes, Alistipes finegoldii, Alistipes onderdonkii, Alistipes putredinis, Anaerocolumna, Anaerocolumna xylanovorans, Anaerostipes, Angelakisella, Angelakisella massiliensis, Bacteroidaceae, Bacteroides, Bacteroides acidifaciens, Bacteroides caecigallinarum, Bacteroides fragilis, Bacteroides rodentium, Bacteroides thetaiotaomicron, Barnesiella, Barnesiella intestinihominis, Barnesiella viscericola, Bifidobacteriaceae, Bifidobacterium, Bifidobacterium anseris, Bifidobacterium italicum, Bifidobacterium longum, Bifidobacterium pseudolongum, Burkholderiales, Catabacter, Catabacter hongkongensis, Chlorflexi, Christensenella, Christensenella minuta, Christensenellaceae, Clostriales, Clostridaceae, Clostridium, Clostridium aldenense, Clostridium asparagiforme, Clostridium cellobioparum, Clostridium cluster XIVa, Clostridium cocleatum, Clostridium hylemonae, Clostridium josui, Clostridium methylpentosum, Clostridium phoceensis, Clostridium populeti, Clostridium saccharogumia, Clostridium saccharolyticum, Clostridium scindens, Clostridium symbiosum, Clostridium xylanovorans, Comamonadaceae, Coprococcus, Coriobactericeae, Culturomica, Culturomica massiliensis, Desulfovibrio, Desulfovibrio fairfieldensis, Desulfovibrionaceae, Dorea, Dorea formicigenerans, Dubosiella, Dubosiella newyorkensis, Eisenbergiella, Eisenbergiella massiliensis, Enterococcus, Enterococcus hirae, Erysipelotrichaceae, Eubacterium, Eubacterium plexicaudatum, Firmicutes, Halovibrio, Halovibrio YL5-2, Lachnospiraceae, Lachnotalea, Lachnotalea glycerini, Lactobacillaceae, Lactobacillus, Lactobacillus hominis, Lactobacillus johnsonii, Lactobacillus reuteri, Massilimaliae, Massilimaliae massiliensis, Mobilitalea, Mobilitalea sibirica, Muribaculum, Muribaculum intestinale, Odoribacteraceae, Olsenella, Olsenella profusa, Olsenella urininfantis, Parabacteroides, Parasutterella, Parasutterella, Parasutterella excrementihominis, Parvibacter, Parvibacter caecicola, Peptostreptococcaceae, Porphyromonadaceae, Prevotellaceae, Prevotellamassilia, Prevotellamassilia timonensis, Proteiniborus, Proteiniborus ethanoligenes, Proteobacteria, Pseudoflavonifractor, Robinsoniella, Robinsoniella peoriensis, Ruminococcaceae, Ruminococcus, Ruminococcus gnavus, Ruthenibacterium, Ruthenibacterium lactatiformans, Sporobacter, Sporobacter termitidis, Subdoligranulum, Tenericutes, and Tyzzerella.

In some embodiments, administering a prebiotic of the disclosure to a subject can increase the abundance of a taxonomic unit (e.g., phylum, class, order, family, genus, species, subspecies, strain, substrain, phylotype, or OTU) associated with or comprising any one or more of Acetatifactor, Acetatifactor muris, Acholeplasma, Acholeplasma pleciae, Acholeplasmataceae, Actinobacteria, Akkermansia, Akkermansia muniniphila, Alistipes, Alistipes finegoldii, Alistipes onderdonkii, Alistipes putredinis, Anaerocolumna, Anaerocolumna xylanovorans, Anaerostipes, Angelakisella, Angelakisella massiliensis, Bacteroidaceae, Bacteroides, Bacteroides acidifaciens, Bacteroides caecigallinarum, Bacteroides fragilis, Bacteroides rodentium, Bacteroides thetaiotaomicron, Barnesiella, Barnesiella intestinihominis, Barnesiella viscericola, Bifidobacteriaceae, Bifidobacterium, Bifidobacterium anseris, Bifidobacterium italicum, Bifidobacterium longum, Bifidobacterium pseudolongum, Burkholderiales, Catabacter, Catabacter hongkongensis, Chlorflexi, Christensenella, Christensenella minuta, Christensenellaceae, Clostriales, Clostridaceae, Clostridium, Clostridium aldenense, Clostridium asparagiforme, Clostridium cellobioparum, Clostridium cluster XIVa, Clostridium cocleatum, Clostridium hylemonae, Clostridium josui, Clostridium methylpentosum, Clostridium phoceensis, Clostridium populeti, Clostridium saccharogumia, Clostridium saccharolyticum, Clostridium scindens, Clostridium symbiosum, Clostridium xylanovorans, Comamonadaceae, Coprococcus, Coriobactericeae, Culturomica, Culturomica massiliensis, Desulfovibrio, Desulfovibrio fairfieldensis, Desulfovibrionaceae, Dorea, Dorea formicigenerans, Dubosiella, Dubosiella newyorkensis, Eisenbergiella, Eisenbergiella massiliensis, Enterococcus, Enterococcus hirae, Erysipelotrichaceae, Eubacterium, Eubacterium plexicaudatum, Firmicutes, Halovibrio, Halovibrio YL5-2, Lachnospiraceae, Lachnotalea, Lachnotalea glycerini, Lactobacillaceae, Lactobacillus, Lactobacillus hominis, Lactobacillus johnsonii, Lactobacillus reuteri, Massilimaliae, Massilimaliae massiliensis, Mobilitalea, Mobilitalea sibirica, Muribaculum, Muribaculum intestinale, Odoribacteraceae, Olsenella, Olsenella profusa, Olsenella urininfantis, Parabacteroides, Parasutterella, Parasutterella, Parasutterella excrementihominis, Parvibacter, Parvibacter caecicola, Peptostreptococcaceae, Porphyromonadaceae, Prevotellaceae, Prevotellamassilia, Prevotellamassilia timonensis, Proteiniborus, Proteiniborus ethanoligenes, Proteobacteria, Pseudoflavonifractor, Robinsoniella, Robinsoniella peoriensis, Ruminococcaceae, Ruminococcus, Ruminococcus gnavus, Ruthenibacterium, Ruthenibacterium lactatiformans, Sporobacter, Sporobacter termitidis, Subdoligranulum, Tenericutes, and Tyzzerella.

In some embodiments, administering a prebiotic of the disclosure to a subject can decrease the abundance of a taxonomic unit (e.g., phylum, class, order, family, genus, species, subspecies, strain, substrain, phylotype, or OTU) associated with or comprising any one or more of Acetatifactor, Acetatifactor muris, Acholeplasma, Acholeplasma pleciae, Acholeplasmataceae, Actinobacteria, Akkermansia, Akkermansia muniniphila, Alistipes, Alistipes finegoldii, Alistipes onderdonkii, Alistipes putredinis, Anaerocolumna, Anaerocolumna xylanovorans, Anaerostipes, Angelakisella, Angelakisella massiliensis, Bacteroidaceae, Bacteroides, Bacteroides acidifaciens, Bacteroides caecigallinarum, Bacteroides fragilis, Bacteroides rodentium, Bacteroides thetaiotaomicron, Barnesiella, Barnesiella intestinihominis, Barnesiella viscericola, Bifidobacteriaceae, Bifidobacterium, Bifidobacterium anseris, Bifidobacterium italicum, Bifidobacterium longum, Bifidobacterium pseudolongum, Burkholderiales, Catabacter, Catabacter hongkongensis, Chlorflexi, Christensenella, Christensenella minuta, Christensenellaceae, Clostriales, Clostridaceae, Clostridium, Clostridium aldenense, Clostridium asparagiforme, Clostridium cellobioparum, Clostridium cluster XIVa, Clostridium cocleatum, Clostridium hylemonae, Clostridium josui, Clostridium methylpentosum, Clostridium phoceensis, Clostridium populeti, Clostridium saccharogumia, Clostridium saccharolyticum, Clostridium scindens, Clostridium symbiosum, Clostridium xylanovorans, Comamonadaceae, Coprococcus, Coriobactericeae, Culturomica, Culturomica massiliensis, Desulfovibrio, Desulfovibrio fairfeldensis, Desulfovibrionaceae, Dorea, Dorea formicigenerans, Dubosiella, Dubosiella newyorkensis, Eisenbergiella, Eisenbergiella massiliensis, Enterococcus, Enterococcus hirae, Erysipelotrichaceae, Eubacterium, Eubacterium plexicaudatum, Firmicutes, Halovibrio, Halovibrio YL5-2, Lachnospiraceae, Lachnotalea, Lachnotalea glycerini, Lactobacillaceae, Lactobacillus, Lactobacillus hominis, Lactobacillus johnsonii, Lactobacillus reuteri, Massilimaliae, Massilimaliae massiliensis, Mobilitalea, Mobilitalea sibirica, Muribaculum, Muribaculum intestinale, Odoribacteraceae, Olsenella, Olsenella profusa, Olsenella urininfantis, Parabacteroides, Parasutterella, Parasutterella, Parasutterella excrementihominis, Parvibacter, Parvibacter caecicola, Peptostreptococcaceae, Porphyromonadaceae, Prevotellaceae, Prevotellamassilia, Prevotellamassilia timonensis, Proteiniborus, Proteiniborus ethanoligenes, Proteobacteria, Pseudoflavonifractor, Robinsoniella, Robinsoniella peoriensis, Ruminococcaceae, Ruminococcus, Ruminococcus gnavus, Ruthenibacterium, Ruthenibacterium lactatiformans, Sporobacter, Sporobacter termitidis, Subdoligranulum, Tenericutes, and Tyzzerella.

In some embodiments, administering a prebiotic of the disclosure increases or decreases the abundance of a microbial taxonomic unit that promotes inflammation. In some embodiments, administering a prebiotic of the disclosure increases or decreases the abundance of a microbial population that reduces inflammation. In some embodiments, administering a prebiotic of the disclosure increases the abundance of a microbial population that is negatively correlated with cancer progression. In some embodiments, administering a prebiotic of the disclosure increases or decreases the abundance of a microbial population is positively correlated with cancer progression.

In some embodiments, administering a prebiotic of the disclosure increases the diversity of glycosyl hydrolases encoded by the microbiota. In some embodiments, administering a prebiotic of the disclosure increases the abundance of glycosyl hydrolases expressed by the microbiota.

In some embodiments, the abundance of one or more microbial taxonomic units is altered by a prebiotic as disclosed herein, and further altered by an additional agent. An additional agent can be, for example, a second prebiotic, a probiotic, or a drug (e.g., an anti-cancer agent, a kinase inhibitor, an immune checkpoint inhibitor, an antibiotic, etc).

### Enhanced Immunity

In some embodiments, administering a prebiotic of the disclosure enhances an immune response in a subject. For example, administering mucin or inulin can result in alterations in the microbiota that potentiate or enhance an anti-tumor immune response.

Enhancing an immune response can comprise enhancing anti-cancer immunity in a subject, for example, by promoting an anti-tumor immune response. Enhancing an anti-tumor immune response can be useful for reducing or ameliorating a cancer in a subject, for example, increasing survival likelihood, preventing or delaying cancer progression, preventing or delaying tumor growth, inducing cancer remission, increasing the likelihood of progression-free survival, or a combination thereof.

In some embodiments, enhancing an immune response comprises enhancing a pro-inflammatory response and/or reducing an anti-inflammatory response. Enhancing a pro-inflammatory response and/or reducing an anti-inflammatory response can be useful, for example, for promoting attack of cancer cells by immune cells. In some embodiments, enhancing an immune response can comprise enhancing an anti-inflammatory response and/or reducing a pro-inflammatory response. Enhancing an anti-inflammatory response and/or reducing a pro-inflammatory response can be useful, for example, for reducing toxicity in a subject.

Enhancing anti-cancer immunity can comprise increasing the infiltration of a subset of immune cells into a tumor, for example, innate immune cells, adaptive immune cells, myeloid immune cells, lymphoid immune cells, CD45+ cells, lymphocytes, T cells, CD4+ T cells, CD8+ T cells, effector T cells (e.g., CD44hi CD4+ or CD8+ T cells), Th1 cells, Th2 cells, Th9 cells, Th17 cells, memory T cells (e.g., central memory T cells, effector-memory T cells, resident memory T cells), tumor-specific T cells, gamma-delta T cells, B cells, antigen-presenting cells, dendritic cells, plasmacytoid dendritic cells, CD8a+ dendritic cells, monocytes, macrophages, neutrophils, natural killer cells, natural killer T cells, innate lymphoid cells, mast cells, or a combination thereof.

In some embodiments, infiltration of a subset of immune cells into a tumor is increased by at least 10%, 20%, 30%, 40%, 50%, 60%, 70%, 80%, 90%, 2.0-fold, 2.1-fold, 2.2-fold, 2.3-fold, 2.4-fold, 2.5-fold, 2.6-fold, 2.7-fold, 2.8-fold, 3.0-fold, 3.1-fold, 3.2-fold, 3.3-fold, 3.4-fold, 3.5-fold, 4-fold, 5-fold, 6-fold, 7-fold, 8-fold, 9-fold, 10-fold, 15-fold, 20-fold, 30-fold, 40-fold, 50-fold, 100-fold or more in a subject administered a prebiotic described herein compared to a subject not administered the prebiotic.

Enhancing anti-cancer immunity can comprise altering expression of an immune system-related gene, for example, increasing or decreasing expression of an immune system-related gene at mRNA and/or protein level.

An immune system-related gene can be a cytokine. Non-limiting examples of cytokines include Interferons (IFNs), Interleukins (ILs), interferon gamma (IFN-g), type I IFNs, IL-1, IL-1a, IL-1b, IL-2, IL-6, IL-10, Il-12, IL-17, IL-17A, IL-23, and TNF-α.

An immune system-related gene can be a chemokine. Non-limiting examples of chemokines include CCL3, CCL4, CCL5, CCL8, CXCL1, CXCL2, CXCL3 and CXCL13.

An immune system-related gene can be a gene involved in antigen presentation or T cell co-stimulation. Non-limiting examples of genes involved in antigen presentation or T cell co-stimulation include an MHC-I gene, an MHC-II gene, an HLA gene, CD40, CD80, CD86, and ICOS.

An immune system-related gene can be a pattern recognition receptor. Non-limiting examples of pattern recognition receptors include toll-like receptors (TLRs), RIG-I-like receptors (RLRs), Nod-like receptors (NLRs), TLR3, TLR7, and NOD2.

An immune system-related gene can encode a product involved in immune signaling and/or immune effector mechanisms. Non-limiting examples of products involved in immune signaling include STAT proteins (e.g., STAT1-6), granzymes, granzyme B, CD107a, and perforin.

In some embodiments, expression of an immune system-related gene is altered systemically, for example, in a subject's blood. In some embodiments, expression of an immune system-related gene is altered locally, for example, in a tumor microenvironment, or in a tumor-draining lymph node. In some embodiments, expression of an immune system-related gene is altered within a cell subset, e.g., within an immune cell subset as disclosed herein. In some embodiments, expression of an immune system-related gene is altered within a cell subset within a tumor microenvironment (e.g., immune cells, cancer cells, or stromal cells). In some embodiments, expression of an immune system-related gene is altered within a cell subset outside of a tumor microenvironment (e.g., in immune cells, epithelial cells, or mucosal cells).

In some embodiments, expression of an immune system-related gene is increased by at least 10%, 20%, 30%, 40%, 50%, 60%, 70%, 80%, 90%, 2.0-fold, 2.1-fold, 2.2-fold, 2.3-fold, 2.4-fold, 2.5-fold, 2.6-fold, 2.7-fold, 2.8-fold, 3.0-fold, 3.1-fold, 3.2-fold, 3.3-fold, 3.4-fold, 3.5-fold, 4-fold, 5-fold, 6-fold, 7-fold, 8-fold, 9-fold, 10-fold, 15-fold, 20-fold, 30-fold, 40-fold, 50-fold, 100-fold or more in a subject administered a prebiotic described herein compared to a subject not administered the prebiotic.

In some embodiments, expression of an immune system-related gene is decreased by at least 10%, 20%, 30%, 40%, 50%, 60%, 70%, 80%, 90%, 2.0-fold, 2.1-fold, 2.2-fold, 2.3-fold, 2.4-fold, 2.5-fold, 2.6-fold, 2.7-fold, 2.8-fold, 3.0-fold, 3.1-fold, 3.2-fold, 3.3-fold, 3.4-fold, 3.5-fold, 4-fold, 5-fold, 6-fold, 7-fold, 8-fold, 9-fold, 10-fold, 15-fold, 20-fold, 30-fold, 40-fold, 50-fold, 100-fold or more in a subject administered a prebiotic described herein compared to a subject not administered the prebiotic.

In some embodiments, an immune response can be enhanced by a prebiotic as disclosed herein, and further enhanced by an additional agent. An additional agent can be, for example, a second prebiotic, a probiotic, or a drug (e.g., an anti-cancer agent, a kinase inhibitor, an immune checkpoint inhibitor, a cell therapy, a CAR-T cell, a transgenic T cell, a chemotherapeutic, etc.).

### Conditions to be Treated, Reduced, Ameliorated, or Prevented

The prebiotics of the disclosure can be used to treat, reduce, or ameliorate a condition in a subject, for example, by altering the gut microbiota of the subject. In some embodiments, administering a prebiotic of the disclosure can alter the gut microbiota of a subject and promote anti-cancer immunity. Examples of conditions that can be treated, reduced, or ameliorated by prebiotics of the disclosure include, but are not limited to, acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, and combinations thereof.

In some embodiments, a prebiotic of the disclosure is used to treat, reduce, ameliorate or prevent melanoma. In some embodiments, a prebiotic of the disclosure is used to treat, reduce, ameliorate or prevent colorectal cancer.

In some embodiments, co-administration of a prebiotic with an additional agent results in an additive therapeutic effect. An additional agent can be, for example, a second prebiotic, a probiotic, or a drug (e.g., an anti-cancer agent, a kinase inhibitor, an immune checkpoint inhibitor, an antibiotic, a chemotherapeutic, a CAR-T cell, a transgenic T cell, etc.). An additive therapeutic effect can be, for example, increasing survival likelihood, preventing or delaying cancer progression, preventing or delaying tumor growth, inducing cancer remission, increasing the likelihood of progression-free survival, or a combination thereof.

### Pharmaceutical Compositions

In some embodiments, the compositions described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of a composition or prebiotic described herein with one or more other chemical components (e.g. pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

The disclosed compositions can optionally comprise pharmaceutically-acceptable preservatives.

The pH of the disclosed composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

### Methods of Administering

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors.

In some embodiments, a prebiotic disclosed herein can be administered to a subject at a dose of, for example, at least 1 mg, at least 5 mg, at least 10 mg, at least 15 mg, at least 20 mg, at least 30 mg, at least 40 mg, at least 50 mg, at least 60 mg, at least 70 mg, at least 80 mg, at least 90 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1 g, at least 1.5 g, at least 2 g, at least 2.5 g, at least 3 g, at least 3.5 g, at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, at least 10 g, at least 10.5 g, at least 11 g, at least 11.5 g, at least 12 g, at least 12.5 g, at least 13 g, at least 13.5 g, at least 14 g, at least 14.5 g, at least 15 g, at least 15.5 g, at least 16 g, at least 16.5 g, at least 17 g, at least 17.5 g, at least 18 g, at least 18.5 g, at least 19 g, at least 19.5 g, at least 20 g, at least 20.5 g, at least 21 g, at least 22 g, at least 23 g, at least 24 g, at least 25 g, at least 26 g, at least 27 g, at least 28 g, at least 29 g, at least 30 g, at least 35 g, at least 40 g, at least 45 g, at least 50 g, at least 55 g, at least 60 g, at least 65 g, at least 70 g, at least 75 g, at least 80 g, at least 85 g, at least 90 g, at least 95 g, at least 100 g, at least 110 g, at least 120 g, at least 130 g, at least 140 g, at least 150 g, at least 160 g, at least 170 g, at least 180 g, at least 190 g, at least 200 g, at least 250 g, at least 300 g, at least 350 g, at least 400 g, at least 450 g, at least 500 g, or more.

In some embodiments, a prebiotic disclosed herein can be administered to a subject at a dose of, for example, at most 1 mg, at most 5 mg, at most 10 mg, at most 15 mg, at most 20 mg, at most 30 mg, at most 40 mg, at most 50 mg, at most 60 mg, at most 70 mg, at most 80 mg, at most 90 mg, at most 100 mg, at most 150 mg, at most 200 mg, at most 250 mg, at most 300 mg, at most 350 mg, at most 400 mg, at most 450 mg, at most 500 mg, at most 550 mg, at most 600 mg, at most 650 mg, at most 700 mg, at most 750 mg, at most 800 mg, at most 850 mg, at most 900 mg, at most 950 mg, at most 1 g, at most 1.5 g, at most 2 g, at most 2.5 g, at most 3 g, at most 3.5 g, at most 4 g, at most 4.5 g, at most 5 g, at most 5.5 g, at most 6 g, at most 6.5 g, at most 7 g, at most 7.5 g, at most 8 g, at most 8.5 g, at most 9 g, at most 9.5 g, at most 10 g, at most 10.5 g, at most 11 g, at most 11.5 g, at most 12 g, at most 12.5 g, at most 13 g, at most 13.5 g, at most 14 g, at most 14.5 g, at most 15 g, at most 15.5 g, at most 16 g, at most 16.5 g, at most 17 g, at most 17.5 g, at most 18 g, at most 18.5 g, at most 19 g, at most 19.5 g, at most 20 g, at most 20.5 g, at most 21 g, at most 22 g, at most 23 g, at most 24 g, at most 25 g, at most 26 g, at most 27 g, at most 28 g, at most 29 g, at most 30 g, at most 35 g, at most 40 g, at most 45 g, at most 50 g, at most 55 g, at most 60 g, at most 65 g, at most 70 g, at most 75 g, at most 80 g, at most 85 g, at most 90 g, at most 95 g, at most 100 g, at most 110 g, at most 120 g, at most 130 g, at most 140 g, at most 150 g, at most 160 g, at most 170 g, at most 180 g, at most 190 g, at most 200 g, at most 250 g, at most 300 g, at most 350 g, at most 400 g, at most 450 g, at most 500 g, or less.

In some embodiments, a prebiotic disclosed herein can be administered to a subject at a dose of, for example, about 1 mg to about 500 g, about 10 mg to about 100 mg, about 50 mg to about 50 g, about 100 mg to about 30 g, about 200 mg to about 20 g, about 300 mg to about 15 g, about 500 mg to about 10 g, about 1 g to about 25 g, about 1 g to about 20 g, about 1 g to about 15 g, about 1 g to about 10 g, about 5 g to about 25 g, about 5 g to about 20 g, about 5 g to about 15 g, about 5 g to about 10 g, about 10 g to about 25 g, about 10 g to about 20 g, about 10 g to about 15 g, about 50 mg to about 900 mg, about 1 mg to about 100 mg, about 100 mg to about 800 mg, about 50 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, about 1 g to about 2 g, about 2 g to about 3 g, about 3 g to about 4 g, about 4 g to about 5 g, about 5 g to about 6 g, about 6 g to about 7 g, about 7 g to about 8 g, about 8 g to about 9 g, about 9 g to about 10 g, about 10 g to about 11 g, about 11 g to about 12 g, about 13 g to about 14 g, about 14 g to about 15 g, about 15 g to about 16 g, about 16 g to about 17 g, about 17 g to about 18 g, about 18 g to about 19 g, about 19 g to about 20 g, or about 20 g to about 25 g.

A prebiotic as disclosed herein can be administered to a subject at any frequency necessary to provide a desired effect, for example, an alteration in the microbiota, an enhanced immune response, enhanced anti-tumor immunity etc. A prebiotic can be administered, for example, monthly, fortnightly, once per week, twice per week, three times per week, four times per week, five times per week, six times per week, daily, two times per day, three times per day, four times per day, five times per day, six times per day, seven times per day, eight times per day, nine times per day, ten times per day, eleven times per day, or twelve times per day.

The compositions described herein can be administered to the subject in a variety of ways, including orally, parenterally, intravenously, intradermally, intramuscularly, colonically, rectally or intraperitoneally. In some embodiments, a prebiotic or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a prebiotic can be in any suitable form for oral administration, such as liquid, tablets, capsules, or the like. The oral formulations can be further coated or treated to prevent or reduce dissolution in stomach. The compositions of the present invention can be administered to a subject using any suitable methods known in the art. Suitable formulations for use in the present invention and methods of delivery are generally well known in the art. For example, a prebiotic described herein can be formulated as pharmaceutical compositions with a pharmaceutically acceptable diluent, carrier or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical compositions described herein are administered orally. In some embodiments, the pharmaceutical compositions described herein are administered topically. In such embodiments, the pharmaceutical compositions described herein are formulated into a variety of topically administrable compositions, such as solutions, suspensions, lotions, gels, pastes, shampoos, scrubs, rubs, smears, medicated sticks, medicated bandages, balms, creams or ointments. In some embodiments, the pharmaceutical compositions described herein are administered topically to the skin. In some embodiments, the pharmaceutical compositions described herein are administered by inhalation. In some embodiments, the pharmaceutical compositions described herein are formulated for intranasal administration. Such formulations include nasal sprays, nasal mists, and the like. In some embodiments, the pharmaceutical compositions described herein are formulated as eye drops. In some embodiments, the pharmaceutical compositions described herein are: (a) systemically administered to the subject; and/or (b) administered orally to the subject; and/or (c) intravenously administered to the subject; and/or (d) administered by inhalation to the subject; and/or (e) administered by nasal administration to the subject; or and/or (f) administered by injection to the subject; and/or (g) administered topically to the subject; and/or (h) administered by ophthalmic administration to the subject; and/or (i) administered rectally to the subject; and/or (j) administered non-systemically or locally to the subject. In some embodiments, the pharmaceutical compositions described herein are administered orally to the subject. In certain embodiments, a composition described herein is administered in a local rather than systemic manner. In some embodiments, a composition described herein is administered with intraperitoneal injection. In some embodiments, a composition described herein is administered topically. In some embodiments, a composition described herein is administered systemically.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Injection can be conducted using sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

In some embodiments, the prebiotics described herein can be used singly or in combination with one or more therapeutic agents as components of mixtures. For example, a prebiotic of the disclosure can be co-formulated or co-administered with other agents, for example, anti-cancer agents.

An anti-cancer agent can be a compound, an antibody, or an antibody fragment, variant, or derivative thereof. In some embodiments, the prebiotics described herein can be used before, during, or after treatment with an anti-cancer agent.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1: Experimental Procedures

Animals and tumor models. All experimental animal procedures were approved by the Institutional Animal Care and Use Committee of Sanford Burnham Prebys Medical Discovery Institute (SBP) and complied with all relevant ethical regulations for animal testing and research. C57BL/6 mice were obtained from Sanford Burnham Prebys Medical Discovery Institute. OT-I mice were bred at SBP to CD45.1 mice (B6.SJLB6.SJL-Ptprc^{a} Pepc^{b}/BoyJ) that were obtained from Jackson Laboratories. C3H/HeOuJ mice were purchased from Jackson laboratories. Male 6-8-week-old mice were used for all experiments. Germ-free ASF-bearing C3H/HeN mice were bred and maintained at the University of Nebraska-Lincoln (UNL) Gnotobiotic Mouse Facility under gnotobiotic conditions in flexible film isolators. Experiments involving GF and gnotobiotic mice were approved by the Institutional Animal Care and Use Committee (IACUC) at UNL. All mice were fed an autoclaved chow diet ad libitum (LabDiet 5K67, Purina Foods). Germ-free status was routinely checked as previously described. Briefly, fresh feces were collected and analyzed by bacterial 16S rRNA gene-specific PCR (30 cycles, universal bacteria primers 8F and 1391R) in combination with aerobic and anaerobic culture of feces in Brain Heart Infusion, Wilkins-Chalgren and Yeast Mold broths, and on Tryptic Soy Agar plates (all media from Difco™ Becton Dickinson) at 37° C. for 7 days. ASF colonization status was verified by qPCR analysis of fecal samples. Briefly, genomic DNA was extracted from fecal samples and ASF bacteria were quantified by qPCR with species-specific primers. Mouse selection for experiments was not formally randomized or blinded. For tumor growth experiments, mice were injected subcutaneously (s.c.) with 1×10⁶ tumor cells. Tumor size was measured twice a week for calculation of tumor volume. Tumors were weighed at the time of excision.

Cell lines and gene silencing. Braf^{V600E/+}; Pten^{-/-}; Cdkn2a^{-/-} mouse melanoma cell line YUMM1.5 was kindly provided by Marcus Bosenberg. MC-38 cell line was kindly provided by Michael Karin. MaN-RASQ^{61K} mouse melanoma cell line was kindly provided by Lionel Larue. SW1 mouse melanoma cells were gift from Margaret Kripke lab. B160VA were obtained from Linda Bradley lab. All cell lines were maintained in Dulbecco's modified Eagle's medium supplemented with 10% fetal bovine serum and antibiotics. All cell lines were free of mycoplasma and were authenticated.

Bacterial strains. The Altered Schaedler Flora consisted of the following 8 isolates: ASF 356, Clostridium sp.; ASF 360, Lactobacillus intestinalis; ASF 361, Lactobacillus murinus; ASF 457, Mucispirillum schaedleri; ASF 492, Eubacterium plexicaudatum; ASF 500, Pseudoflavonifractor sp.; ASF 502, Clostridium sp.; and ASF 519, Parabacteroides goldsteinii.

Anaerobic fecal cultures. Stool collected from 12 healthy vegetarian participants were inoculated (approximately 10⁶ cells) into a chemically defined medium (CDM), or CDM supplemented with either 1% inulin or 1% porcine gastric mucin in Hungate tubes. Anaerobic cultures (9% H2, 81% N2) were grown statically for 3-4 days at 37° C. and grown to approximate saturation.

Chemically-defined medium (CDM). CDM contains 50 mM HEPES, 2.2 mM KH₂PO₄, 10 mM Na₂HPO₄, 60 mM NaHCO₃, 4 mM of each amino acid, except leucine (15 mM), 10 mL ATCC, Trace Mineral Supplement. CDM contained nucleoside bases (100 mg/L), inosine, xanthine, adenine, guanine, cytosine, thymidine and uracil (400 mg/L). CDM contained choline (100 mg/L), ascorbic acid (500 mg/L), lipoic acid (2 mg/L), hemin (1.2 mg/L) and myo-inositol (400 mg/L). Resazurin (1 mg/L) was added to visually monitor dissolved oxygen. The pH of the media was adjusted to 7.4. The 2×CDM and medicinal herbs (powder) in sterile water (2%) were separately reduced in an anaerobic chamber (Coy Labs) for 3 days.

Bacterial DNA extraction and 16S library preparation. Mouse microbiota displays a stable homeostatic state when they are around 8 weeks. In following this protocol, microbiota was not collected earlier than 8 weeks. Mouse fecal pellets were frozen on dry ice, and stored at −80° C. Bacterial DNA was extracted using the QIAmp Fast DNA Stool Mini Kit (Qiagen). To ensure efficient cell lysis, a 5-min bead-beating step using a Mini-Beadbeater-16 was included (Biospec Products, OK, USA). Library preparation for the Illumina MiSeq platform was performed by amplification of the V3-V4 region of the bacterial 16S ribosomal DNA gene using

Forward primer:
5′-TCGTCGGCAGCGTCAGATGTGTATAAGAGACAGCCTACGGGNGGCW
GCAG
and
Reverse primer:
5′-GTCTCGTGGGCTCGGAGATGTGTATAAGAGACAGGACTACHVGGGT
ATCTAATC

C. Adapter and barcode sequences for dual indices were used as described by Illumina. PCR clean up steps were performed with QIAquick 96-PCR Clean up kit (Qiagen, Germany), and library quantification was performed using a KAPA Library Quantification Kit for Illumina platforms (KAPA Biosystems, MA, USA). An Experion Automated Gel Electrophoresis System (Bio-Rad, CA, USA) was used to measure the DNA concentration and purity of the pooled libraries. The 16S libraries were sequenced at Novagene (Beijing, China) and the SBP sequencing Core.

16S sequencing data processing. The original FASTQ files from Illumina 250 basepair paired-end sequencing of the samples were processed using a novel 16S amplicon sequencing pipeline HiMap (http://github.com/taolonglab/himap; BioRxiv 565572). The output of HiMap is Operational Strain Unit (OSU) which contains one or more bacterial strains that best match the 16S sequence and cannot be further distinguished. The percentage similarity between the 16S sequence and the aligned region of 16S rRNA genes of the strains in the OSU is indicated. OSUs mapped to the same strains are grouped together (adding read counts) if percentage similarities are within 3%. Read counts are converted into relative abundance as described in HiMap. Log 10-transformed relative abundances were used for comparisons between samples under different experimental conditions.

Taxa selection. Taxa that distinguished inulin or mucin treated-mice microbiota from control mice were selected based on the following three sets: (1) Taxa induced by inulin or mucin were selected by performing a paired one-tail Wilcox rank sum test on the log 10 transformed relative abundances of all OSU groups in mice treated with prebiotics at time point B (after prebiotics treatment and before tumor injection) compared with time point A (before prebiotics treatment) with abundance at time point B greater than time point A. Taxa with p-values less than 0.05 were selected as set 1. (2) A similar paired one-tail Wilcox rank sum test on the log 10 transformed relative abundances of all OSU groups in mice in the control group at time point B compared with time point A. Taxa with p-values less than 0.2 were selected as set 2. This set contains taxa that were induced in the control group from timepoint A to timepoint B. (3) The third set of OSU groups were selected by calculating spearman correlation between each of the OSU groups and tumor size at time point B and keeping OSU groups with p-value less than 0.1. Final set of prebiotics (inulin or mucin) induced taxa are the difference between set 1 and set 2 intersected with set 3. These are the taxa induced by inulin or mucin, but not in the control group, and their relative abundances before tumor injection are negatively correlated with tumor size at tumor collection. For analysis of MEKi combination prebiotics, OSU groups in the fecal samples of mice treated with prebiotics (inulin or mucin) in combination with MEKi or treated with prebiotics alone were compared at time point D (final time point before tumor collection) with unpaired two-sided Wilcox rank sum test. OSU groups with p-value less than 0.05 were selected for calculating spearman correlations with tumor size at time point D. The OSU groups with correlation p-value less than 0.1 were kept as the differential taxa that are negatively correlated with tumor size.

Tumor digestion. Tumors were excised, minced, and digested with 1 mg/ml collagenase D (Roche) and 100 μg/ml DNase I (Sigma) at 37° C. for 1 h. Digests were then passed through a 70-μm cell strainer to generate a single-cell suspension. The cells were washed twice with PBS containing 2 mM EDTA, and then stained for flow cytometry.

Flow cytometry. Tumor-derived single-cell suspensions were washed twice with FACS staining buffer, fixed for 15 min with 1% formaldehyde in PBS, washed twice, and resuspended in FACS staining buffer. For intracellular cytokine staining, cells were resuspended in complete RPMI-1640 (containing 10 mM HEPES, 1% non-essential amino acids and L-glutamine, 1 mM sodium pyruvate, 10% heat-inactivated fetal bovine serum (FBS), and antibiotics) supplemented with 50 U/mL IL-2 (NCI), 1 mg/mL brefeldin A (BFA, Sigma), and incubated with phorbol myristate acetate (10 ng/ml) and ionomycin (0.5 μg/ml) at 37° C. The cells were then fixed and permeabilized using a Cytofix/Cytoperm Kit (BD Biosciences) before staining. Antibodies to the following proteins were used: CD45.2 (104), CD8a (53-6.7), CD4 (GK1.5), CD44 (IM7), TNF-α (MP6-XT22), IFN-γ (XMG1.2), CD11c (N418), CD11b (M1/70), MHC class II (M5/114.15.2), PDCA (129c1), and B220 (RA3-6B2) from BioLegend, and antibodies to IL-2 (JES6-5H4) and MHC class I (AF6-88.5.5.3) from eBioscience. All data were collected on an LSRFortessa (BD Biosciences) and analyzed using FlowJo Software (Tree Star).

Mucin and inulin treatment. For mucin treatment, C57BL/6 mice were subjected to control or water supplemented with 3% mucin (Lee Biosolutions) prior to (14 days) and during tumor inoculation. Water was changed every other day. For inulin treatment, mice were received a diet (TD. 94048, AIN-93M, Purified Diet, ENVIGO) enriched with long-chain inulin by substituting all of sucrose and 5% of corn starch in the control diet or a modified diet (TD. 160256, Modified AIN-93M, Diet w 15% inulin, ENVIGO) prior to (14 days) and during tumor inoculation. Diets were changed 2 times a week.

RNA extraction and qRT-PCR analyses. Total RNA was extracted from tumor samples individually using the RNeasy Fibrous Tissue Midi kit (QIAGEN) or cells treated as indicated using High Capacity Reverse Transcriptase kits (Invitrogen) according to the manufacturer's protocol. Purity and concentration of extracted RNA were checked and quantified by reading at 260 and 280 nm in a NanoDrop spectrophotometer (Thermo Fisher). The qRT-PCR analyses were performed using Syber Green RT-PCR kits (Invitrogen) on a Bio-Rad CFX Connect Real-Time system. Expression levels normalized to 18S or Tubb5 controls. Sequence-specific primers used in this study are shown in TABLE 1.

TABLE 1

| Gene | Forward (5′-3′) | Reverse (5′-3′) |
|---|---|---|
| CCL3 | TTCTCTGTACCATGACACTCTGC | CGTGGAATCTTCCGGCTGTAG |
| CCL4 | TTCCTGCTGTTTCTCTTACACCT | CTGTCTGCCTCTTTTGGTCAG |
| CCL5 | GCTGCTTTGCCTACCTCTCC | TCGAGTGACAAACACGACTGC |
| CCL8 | TCTACGCAGTGCTTCTTTGCC | AAGGGGGATCTTCAGCTTTAGTA |
| TLR7 | ATGTGGACACGGAAGAGACAA | GGTAAGGGTAAGATTGGTGGTG |
| TLR3 | GTGAGATACAACGTAGCTGACTG | TCCTGCATCCAAGATAGCAAGT |
| CD40 | GCTGTGAGGATAAGAACTTGGAG | CTGGTTCGACAGGGCTGAA |
| Stat1 | CGGAGTCGGAGGCCCTAAT | ACAGCAGGTGCTTCTTAATGAG |
| ICOS1 | ATGAAGCCGTACTTCTGCCG | CGCATTTTTAACTGCTGGACAG |
| TNF-α | CCCTCACACTCAGATCATCTTCT | GCTACGACGTGGGCTACAG |
| IL-6 | TAGTCCTTCCTACCCCAATTTCC | TTGGTCCTTAGCCACTCCTTC |
| CXCL2 | CCAACCACCAGGCTACAGG | GCGTCACACTCAAGCTCTG |
| IFN-r | CACGGATAAAACGACCATAGGTG | TCTTGACCTGTCATTTTGCCAG |
| GranzymB | CCACTCTCGACCCTACATGG | GGCCCCCAAAGTGACATTTATT |
| 18S | GTAACCCGTTGAACCCCATT | CCATCCAATCGGTAGTAGCG |
| Tubb 5 | GATCGGTGCTAAGTTCTGGGA | AGGGACATACTTGCCACCTGT |

Bone Marrow-Derived Dendritic Cells (BMDCs). Bone marrow cells were isolated from the tibiae and femurs of C57BL/6 mice treated with or without mucin or inulin and cultured in DMEM medium containing 10% FBS, 1% penicillin/streptomycin, and recombinant mouse GM-CSF (20 ng/ml; BioLegend) for 8 days at 37° C.

Isolation of intestinal epithelial cells. A 10 cm section of mouse small intestine was opened longitudinally, minced, washed in 150 mM NaCl containing 1 mM DTT, and then resuspended in dissociation buffer (130 mM NaCl, 10 mM EDTA, 10 mM Hepes [pH 7.4], 10% FCS, and 1 mM DTT). The sections were incubated at 37° C. for 30 min with vigorous shaking to release the epithelial cells from the lamina propria. The epithelial cell suspension was then carefully aspirated, centrifuged, and washed in ice-cold PBS.

Serum cytokine and chemokine detection. Cytokines and chemokines in the sera of naïve or tumor-bearing mice treated with or without mucin were quantified using the LEGENDplex™ mouse inflammation panel and mouse proinflammatory chemokine panel (BioLegend), respectively. All data were collected on an LSRFortessa (BD Biosciences) and analyzed using LEGENDplex™ software (BioLegend).

In vivo antibody treatments. For anti-PD-1 antibody treatment, mice were injected i.p. with 200 μg anti-PD-1 (clone RMP1-14), or rat IgG2a isotype control on days 7, 10, 13, and 16 after tumor inoculation. All mAbs for in vivo use were GoInVivo™ grade from BioLegend (San Diego, Calif., USA).

In vivo OT-I T cell proliferation assay. CD8+ T cells were isolated from the spleens of naïve OT-I CD45.1+ mice, labeled with CFSE, and injected i.v. into WT (CD45.2) mice treated with or without mucin. After 24 h, the mice were injected s.c. with 1×10⁶ B16-OVA melanoma cells and the mice were left for 7 days. The spleen, tumor-draining lymph nodes, and non-draining lymph nodes were harvested and analyzed by flow cytometry. The proliferation of OT-1 CD8+ T cells was assessed by analysis of CFSE dilution within the population by gating on CD45.1+CD8+ T cells.

CD8+ T cell enrichments. CD8+ T cells were negatively enriched (Stemcell Technologies) from spleens of C57BL/6 mice that were untreated or were treated with mucin or inulin for 2 weeks.

Statistical analysis. Unless otherwise noted, all data are shown as the mean s.e.m. Before statistical analysis, data were subjected to the Kolmogorov-Smirnov test to determine distribution. Variance similarity was tested using an F test for two groups and Bartlett's test for multiple groups. Two groups were compared using the two-tailed t-test for parametric data or the Mann-Whitney U test for non-parametric data. Multiple groups were compared using one-way ANOVA with Tukey's, Dunnett's, or Bonferroni's correction for parametric data or using the Kruskal-Wallis test with Dunn's correction for non-parametric data. Tumor growth curves were analyzed using two-way ANOVA with Sidak's, Tukey's, or Bonferroni's correction for multiple comparisons.

### Example 2: Prebiotics that Enrich for Anti-Tumor Promoting Taxa In Vitro

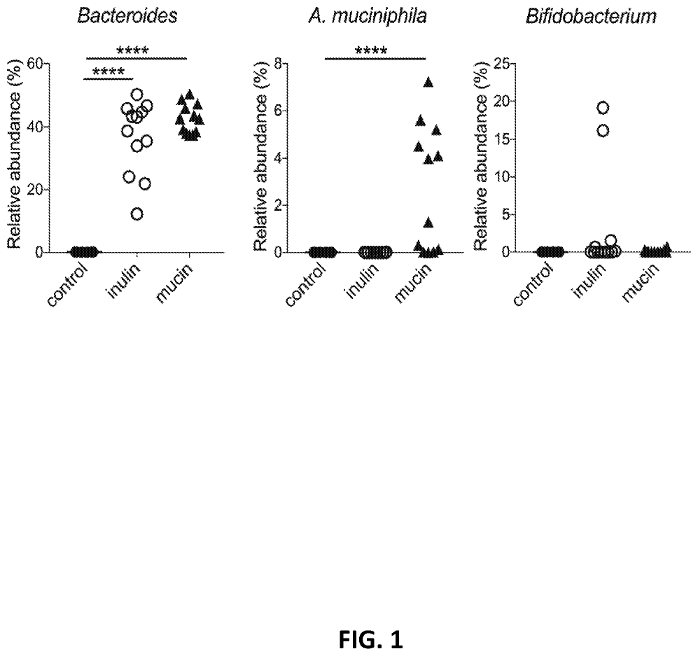

This example demonstrates the effects of inulin and mucin on anaerobic fecal cultivation in vitro. Fecal samples derived from 12 healthy, vegetarian human subjects were cultivated in a chemically-defined medium the presence or absence of 1% prebiotic. Anaerobic cultures (9% H₂, 81% N₂) were grown statically for 3-4 days at 37° C. and grown to approximate saturation. Both inulin and mucin increased the relative abundance of Bacteroides spp., whereas only mucin increased the relative abundance of A. muciniphila in most of the cultures (FIG. 1). Surprisingly, inulin, but not mucin, led to a strong increase of Bifidobacterium spp. in two of these cultures. These results suggested that mucin and inulin can alter the relative abundance of bacterial taxa, including taxa that may affect anti-tumor immunity.

### Example 3: Administration of Mucin or Inulin Reduces Tumor Growth and Induces Anti-Tumor Immunity

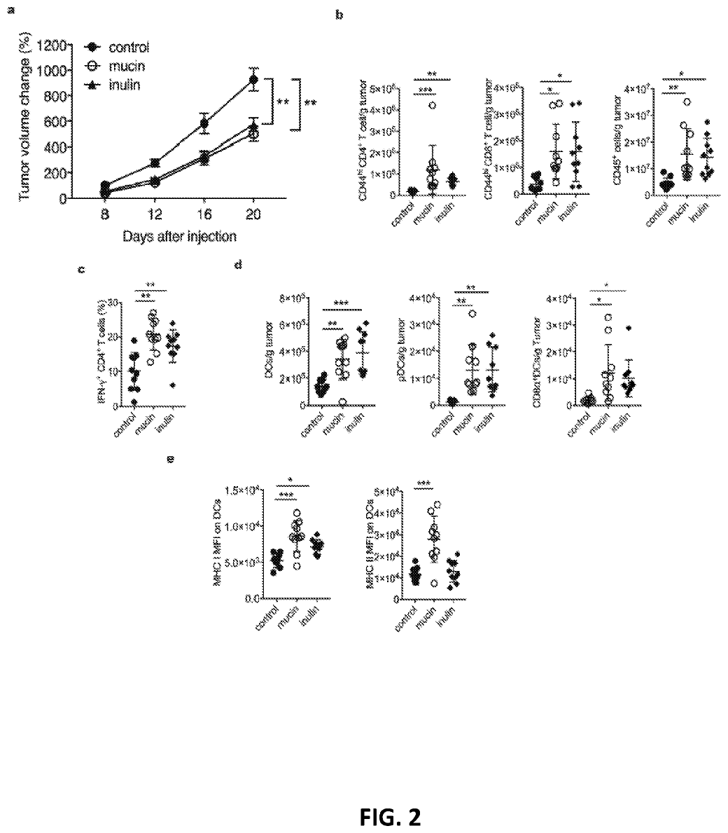

To determine whether prebiotics inhibit tumor growth, mucin (3% in drinking water) or inulin-supplemented chow (15% w/w) were administered to C57BL/6 mice, 2 weeks prior to inoculation of melanoma tumor cells (Yumm1.5; 1×10⁶ cells). The administration of mucin or inulin led to attenuated melanoma tumor growth (FIG. 2A). To determine whether these changes could be attributed to anti-tumor immunity tumor-infiltrating lymphocytes (TILs) were analyzed 20 days following tumor inoculation. Compared to control mice, mucin and inulin-treated mice exhibited an enrichment of effector (CD44hi) CD4+ and CD8+ T cells, and CD45+ cells in tumors (FIG. 2B). Prebiotic treatment also increased the numbers of tumor-infiltrating CD4+ T cells that displayed greater effector function, reflected by elevated IFN-g production (FIG. 2C). A number of dendritic cells (DCs) subsets including plasmatoid DCs (pDCs) as well as CD8a+ conventional DCs were increased in tumors from mucin and inulin-treated mice (FIG. 2D). Further, tumor-resident DCs derived from inulin and mucin-treated mice expressed higher levels of MHC class I as well as MHC class II (FIG. 2E), implying greater stimulatory capacity. These data indicate a prebiotic-induced shift to a pro-inflammatory tumor microenvironment in treated mice that is associated with a more potent anti-tumor response.

### Example 4: Enhanced Intra-Tumoral Expression of Immune Genes in Mucin or Inulin Treated Mice

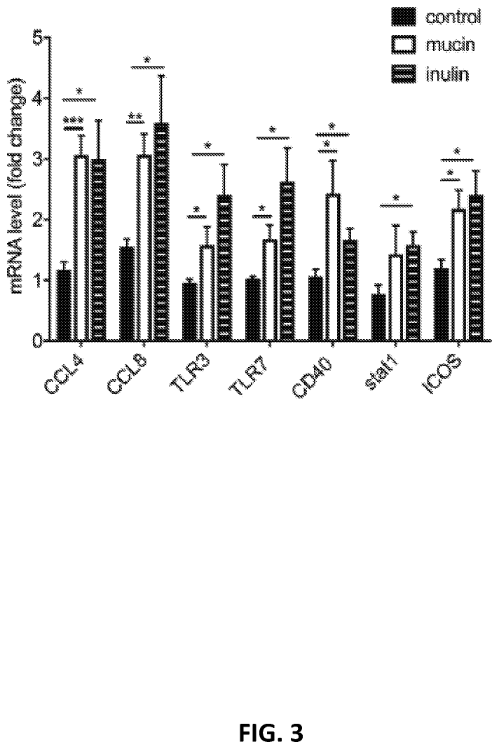

To identify possible mechanisms involved in the greater immune cell infiltration and overall anti-tumor immunity observed in prebiotic-treated mice, changes in the transcription of genes implicated in chemotaxis, immune signaling, and antigen presentation were assessed in tumors. Both prebiotics led to an increased expression of chemokines (CCL4 and CCL8), pattern recognition receptors (TLR3 and TLR7), and antigen presentation (CD40, Stat1 and ICOS) related genes (FIG. 3). These finding suggest that inulin and mucin affect cellular pathways that culminate in induced transcription of genes implicated the recruitment of immune cells as well as in antigen presentation and better tumor recognition by the immune system.

### Example 5: Enhanced Recruitment of Tumor-Specific CD8+ T Cells to Tumor-Draining Lymph Nodes

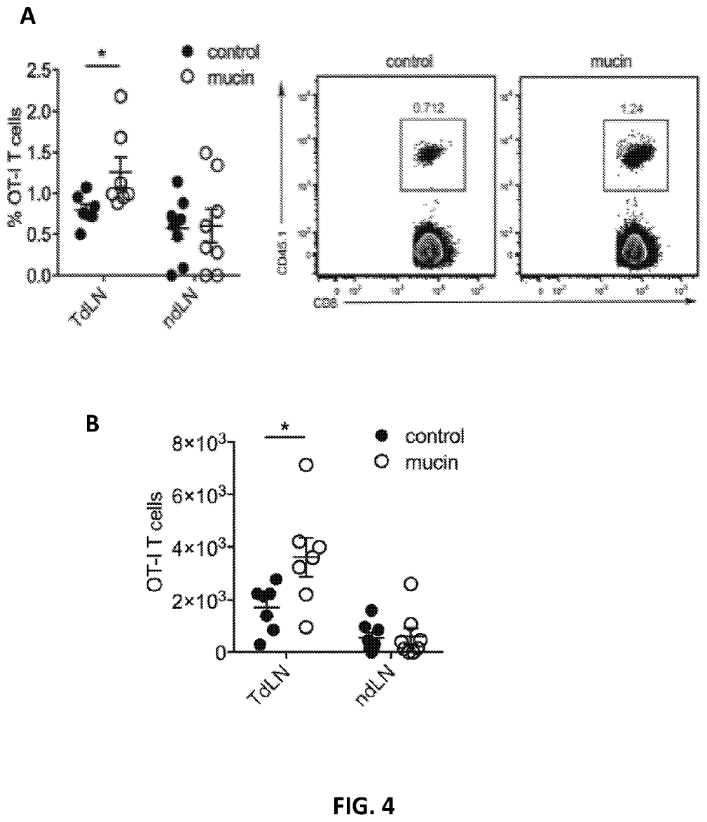

To assess the activation and homing of tumor-specific T cells in prebiotic-treated mice, an adoptive transfer model was used with OVA-specific OT-I transgenic T cells. OVA-specific OT-I CD8+, CD45.1+ T cells were transferred into untreated or mucin-treated WT mice. Mice were injected with OVA-expressing B16F10 tumor cells, and the frequency of OVA-specific OT-I T cells was monitoring in tumor draining and non-tumor-draining lymph nodes. OT-I CD8+ T cells were more abundant in the tumor-draining lymph nodes of mucin-treated mice, compared with control mice (FIG. 4A-B).

### Example 6: Prebiotics Alter Levels of Serum Cytokines and Chemokines

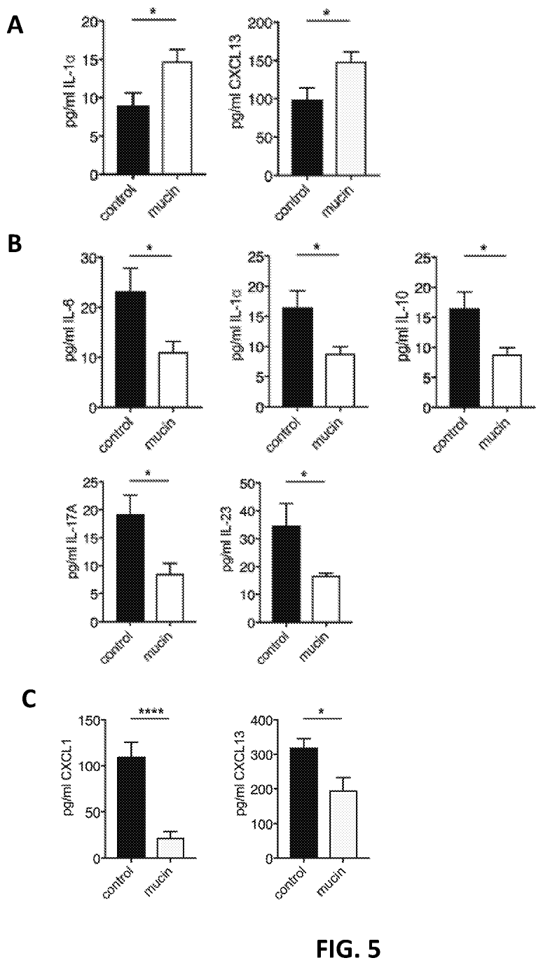

Levels of circulating cytokines and chemokines were quantified in mucin-treated mice both before and after tumor inoculation. Higher levels of IL-1a and CXCL13 were observed in the sera of mice that were subjected to mucin feeding for two weeks prior to tumor inoculation (FIG. 5A). Strikingly, the profile of inflammatory mediators changed following tumor inoculation, where mucin treated mice exhibited reduced levels of IL-6, IL-1a, IL-10, IL-17A, and IL-23 compared with control-treated animals (FIG. 5B). Consistent with these observations, higher levels of IL-6 and IL-17 have been associated with poor clinical outcome, while reduced IL-1a levels have been associated with attenuated tumor growth. Lower serum levels of the chemokines CXCL1 and CXCL13 were also found in mucin-treated, tumor-inoculated mice, compared with control mice (FIG. 5C).

### Example 7: Inulin and Mucin Alter the Gut Microbiota

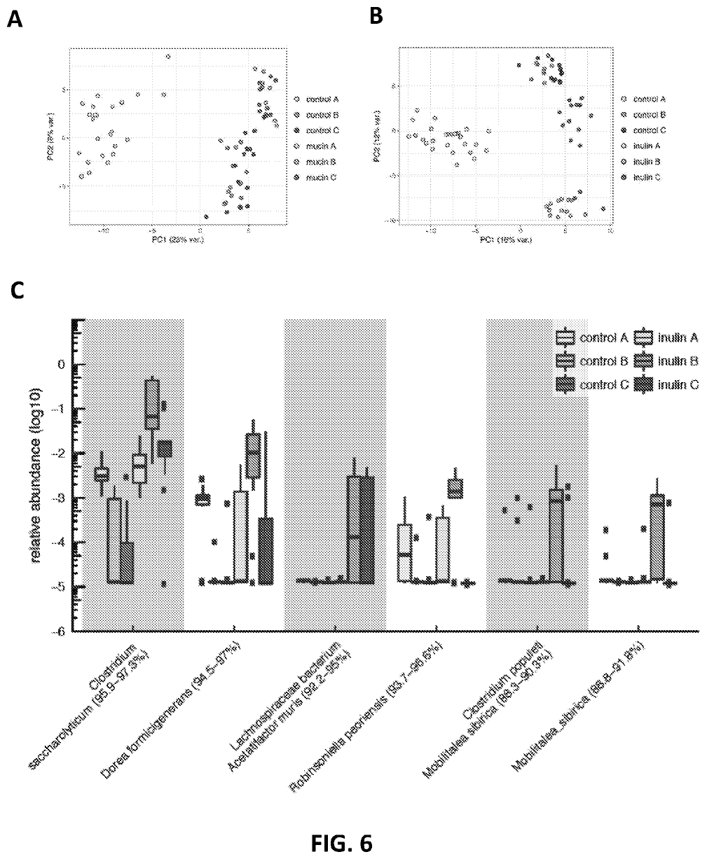

16S rRNA amplicon sequencing was used to profile the fecal microbiota of mice: (i) prior to prebiotic feeding, (ii) 14 days after prebiotic feeding, and (iii) 20 days post-tumor cell inoculation, with or without prebiotic feeding. While mouse gut communities at baseline were heterogeneous and generally not well clustered (FIG. 6A-B), communities formed tighter clusters that were distinct from control mice following prebiotic feeding. These data are consistent with recent observations that distal tumor growth results in a reconfiguration of gut microbiota; alterations in microbiota composition were seen following the introduction of a custom diet in control mice and by the prebiotics used here. Common changes in individual phylotype groups (two or more highly related but not identical 16S sequences of strains approximating a species) from pre- to post-prebiotic treatment, seen in both animal cohorts, were attributed to diet and were not further assessed. Conversely, phylotypes that were associated with a specific prebiotic treatment were subjected to further analysis.

Sequencing of the amplified 16S V3-V4 region followed by computational analysis, led to the identification of phylotype groups that distinguished the microbiota of inulin-treated mice (TABLE 2) and phylotype groups that distinguished the microbiota of mucin-treated mice (TABLE 3) from control mice. Inulin induced an increase in taxa that are phylogenetically coherent, 66% of which map most closely to members of Clostridium cluster XIVa, primarily, Clostridium populeti and Clostridium saccharolyticum (TABLE 2). Although Clostridium cluster XIVa is known to consist of numerous butyrate producers, the phylogenetic distance of the phylotypes profiled here is likely to exclude butyrate as a driver of the anti-tumor phenotype identified herein. Among the phylotypes that displayed increased relative abundance following inulin treatment, 6 were negatively correlated with tumor size (FIG. 6C). Mucin also predominantly enriched taxa with similarity to members of Clostridium cluster XIVa (TABLE 3). None of the phylotypes induced by mucin were negatively correlated with tumor size. These finding suggest that inulin and mucin drive distinct changes in gut microbiota that are capable of inducing anti-tumor immunity.

TABLE 2 provides the abundance of phylotype groups (OSU groups) prior to inulin feeding (A), 14 days after inulin feeding (C), and 20 days post-tumor cell inoculation with inulin feeding (C). P-values were calculated using paired one-tail Wilcoxon rank sum test.

TABLE 2

| | Abundance | | | p-value | |
|---|---|---|---|---|---|
| BLAST best hit/s (% identity) | A | B | C | A vs B | B vs C |
| Enterorhabdus mucosicola (98) | 0.039 | 0.135 | 0.111 | 0.022 | |
| Flintibacter butyricus (99) | 0.003 | 0.024 | 0.012 | 0.017 | |
| Actualibacter muris (100) | 0.015 | 0.184 | 0.042 | 0.0002 | 0.002 |
| Neglecta timonensis (98) | | | | | |
| Parvibacter caecicola (100) | 0.048 | 0.181 | 0.014 | 0.0004 | 0.00003 |
| Adlercreutzia equolifaciens (96) | | | | | |
| Muribaculum intestinale (93) | 0.002 | 0.008 | | 0.048 | |
| Bacteroides acidifaciens (90) | 0.001 | 0.012 | 0.001 | 0.018 | 0.014 |
| Oscillibacter valericigenes (96) | 2.059 | 6.766 | 1.386 | 0.0003 | 0.00003 |
| Murimonas intestini (97) | 0.701 | 16.756 | 3.131 | 0.005 | 0.018 |
| Clostridium celerecrescens (96) XIVa | | | | | |
| Lachnoclostridium pacaense (96) | 0.331 | 9.675 | 1.749 | 0.002 | 0.010 |
| R. lactis (95) | | | | | |
| Eisenbergiella massiliensis (95) | | | | | |
| Lachnoclostridium pacaense (95) | 0.167 | 6.249 | 1.455 | 0.046 | |
| Olsenella profusa (95) | 0.077 | 0.949 | 2.994 | 0.002 | |
| Clostridium xylanolyticum (95) | 0.399 | 2.221 | 1.025 | 0.026 | |
| Lachnoclostridium pacaense (98) | 0.063 | 0.907 | | 0.040 | |
| Clostridium lactatifermentans (95) | 0.785 | 1.770 | 1.346 | 0.038 | |
| C. propionicum (93) | | | | | |
| Dorea formicigenerans (97) | 0.097 | 1.439 | 0.257 | 0.006 | 0.025 |
| C. clostridioforme (96) | | | | | |
| Flintibacter butyricus (92) | 0.001 | 0.004 | | 0.040 | |
| Oscillibacter valericigenes (94) | 0.002 | 0.014 | 0.001 | 0.031 | 0.024 |
| Acetatifactor muris (95) | 0.001 | 0.151 | 0.118 | 0.030 | |
| Robinsoniella peoriensis (97) | 0.018 | 0.144 | 0.001 | 0.002 | 0.001 |
| Ihubacter massiliensis (92) | 0.013 | 0.064 | 0.122 | 0.017 | |
| Emergencia | | | | | |
| Anerostipes | | | | | |
| Eisenbergiella | | | | | |
| Lachno | | | | | |
| Flintibacter butyricus (98) | 0.036 | 0.126 | | 0.049 | |
| Kineothrix alysoides (97) | 0.002 | 0.018 | | 0.046 | |
| C. symbiosum (96) | | | | | |
| Clostridium polysaccharolyticum (87) | 0.060 | 0.298 | 0.008 | 0.001 | 0.00003 |
| C. populeti (87) | | | | | |
| Anaerotaenia torta (87) | 0.031 | 0.073 | | 0.041 | |
| Anaerostipes butyricus (86) | | | | | |
| Clostridium populeti (88) | 0.019 | 0.104 | 0.028 | 0.013 | 0.034 |
| Eisenbergiella massiliensis (88) | | | | | |
| chimera | | | | | |
| Clostridium populeti (88) | 0.001 | 0.107 | 0.020 | 0.008 | 0.035 |
| Eisenbergiella massiliensis (88) | | | | | |
| chimera | | | | | |
| Bacteroides stercoris (90) | 0.005 | 0.076 | 0.014 | 0.002 | 0.011 |
| chimera | | | | | |
| Anaerostipes caccae (87) | 0.004 | 0.118 | 0.004 | 0.020 | 0.020 |
| Murimonas intestini (87) | | | | | |
| Mucispirillum schaedleri (100) | 0.001 | 0.034 | 0.003 | 0.048 | |
| chimera | | | | | |
| Eisenbergiella massiliensis (88) | 0.010 | 0.104 | 0.010 | 0.007 | 0.008 |
| Eisenbergiella massiliensis (91) | 0.003 | 0.067 | 0.006 | 0.004 | 0.007 |
| Anaerostipes hadrus (88) | 0.001 | 0.011 | | 0.039 | |
| Clostridium celecrescens (96) | 0.001 | 0.019 | | 0.041 | |
| Flintibacter butyricus (98) | 0.036 | 0.109 | 0.001 | | 0.014 |
| Pseudoflavonifractor | | | | | |
| C. viride | | | | | |
| Anaerostipes hadrus (85) | 0.038 | 0.048 | 0.008 | | 0.003 |
| Anaerostipes butyricus (86) | 0.031 | 0.064 | 0.010 | | 0.004 |
| Oribacterium sinus (87) | 0.005 | 0.023 | 0.001 | | 0.018 |
| Clostridium populeti (87) | | | | | |
| Clostridium scindens (98) | 0.084 | 0.086 | 0.001 | | 0.022 |
| R. gnavus (98) | | | | | |
| Dorea longicatena (96) | | | | | |
| C. oroticum (96) | | | | | |
| Lachnoclostridium pacaense (98) | 0.063 | 0.786 | 0.014 | | 0.047 |
| C. aldenense (97) | | | | | |
| Christensenella minuta (87) | 0.135 | 0.218 | 0.008 | | 0.019 |
| Christensenella massiliensis (87) | | | | | |

TABLE 3 provides the abundance of phylotype groups (OSU groups) prior to mucin feeding (A), 14 days after mucin feeding (B), and 20 days post-tumor cell inoculation with mucin feeding (C). P-values were calculated using paired one-tail Wilcoxon rank sum test.

TABLE 3

| | Abundance | | | p-value | |
|---|---|---|---|---|---|
| BLAST best hit/s (% identity) | A | B | C | A vs B | B vs C |
| Clostridium scindens (98) | 0.114 | 0.564 | 0.593 | 0.007 | |
| R. gnavus (98) | | | | | |
| Dorea longicatena (96) | | | | | |
| C. oroticum (96) | | | | | |
| Kineothrix alysoides (99) | 6.676 | 15.996 | | 0.024 | |
| C. saccharolyticum (98) | | | | | |
| Kineothrix alysoides (96) | 0.215 | 0.577 | | 0.048 | |
| Murimonas intestini (96) | | | | | |
| C. asparagiforme (95) | | | | | |
| Lachnoclostridium pacaense (96) | 0.346 | 0.960 | | 0.044 | |
| R. lactis (95) | | | | | |
| Eisenbergiella massiliensis (95) | | | | | |
| Anaerotaenia torta (96) | 0.431 | 2.339 | | 0.001 | |
| C. xylanolyticum (95) | | | | | |
| Roseburia faecis (95) | 0.224 | 1.360 | 1.233 | 0.034 | |
| Ihubacter massiliensis (98) | 0.066 | 1.531 | 2.350 | 0.005 | |
| E. timonensis (96) | | | | | |
| Clostridium cellulovorans (89) | 0.047 | 2.208 | | 0.021 | |
| Christensenella minuta (89) | | | | | |
| Clostridium oroticum (98) | 0.049 | 0.209 | 0.101 | 0.050 | |
| Pseudoflavonifractor capillosus (97) | 0.177 | 1.050 | | 3 × 10⁻⁶ | |
| Gracilibacter thermotolerans (88) | 0.083 | 0.187 | | 0.039 | |
| C. propionicum (88) | | | | | |
| Aminipila butyrica (89) | 0.003 | 0.160 | 0.109 | 0.0003 | |
| Emergencia timonensis (87) | | | | | |
| Harryflintia acetispora (93) | 0.007 | 0.019 | 0.094 | | 0.032 |
| Anaerotruncus rubiinfantis (90) | | | | | |
| Acetatifactor muris (92) | 0.871 | 1.280 | 4.419 | | 0.004 |
| Lachnoclostridium pacaense (92) | | | | | |

### Example 8: Overcoming MEK Inhibitor Resistance in Melanoma Via Combination with Inulin

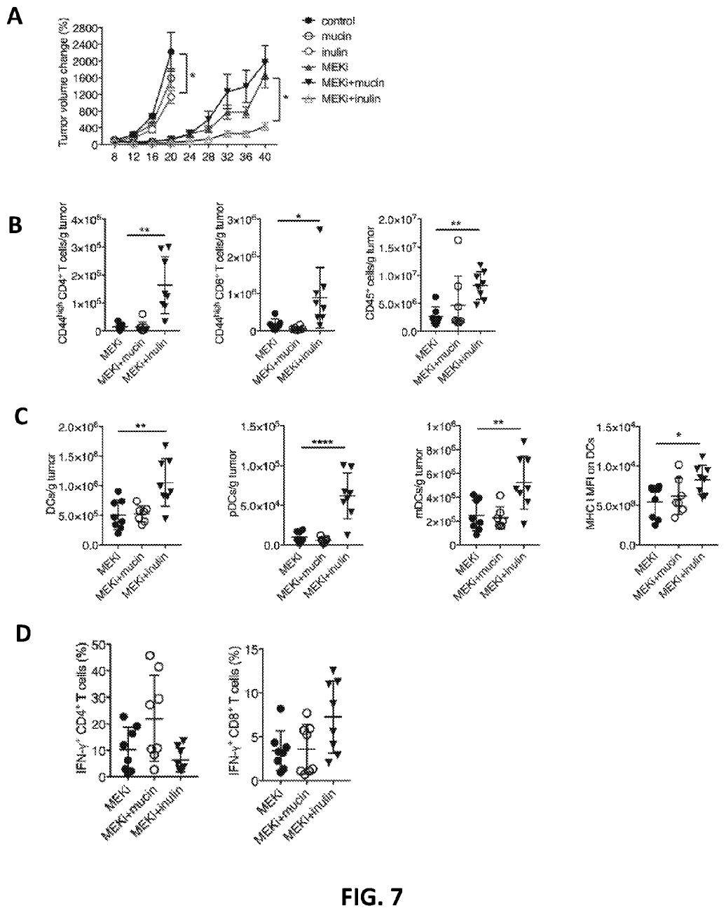

Experiments were conducted to determine whether prebiotics impacted the effectiveness of MEK inhibitor (MEKi) treatment on tumor growth control and treatment resistance. N-Ras mutant melanoma tumor cells (MaNRAS1) were inoculated in mice that were fed with inulin or mucin in combination with (or without) MEKi. In the absence of MEKi, inulin but not mucin, modestly controlled N-Ras mutant tumors (FIG. 7A). Strikingly, the combination of inulin with MEKi revealed an additive effect that was reflected in better tumor growth inhibition (FIG. 7A). Notably, the intrinsic resistance of MaN-Ras melanoma cells to MEKi was delayed in inulin-fed mice (FIG. 7A), implying that MEKi-resistance may be partially overcome by this prebiotic. Consistent with these findings, increases in CD4+ and CD8+ T cells, CD45+ cells and DCs, including pDCs and mDCs, as well MHC-I expression on DCs, were identified in tumors derived from the combination of inulin and MEKi treatment (FIG. 7B-C). However, no differences in cytokine production by T cells were found (FIG. 7D).

### Example 9: Prebiotic-Induced Alterations in Microbiota Associated with Control of N-Ras Melanoma Tumors and Overcoming MEK Inhibitor Resistance

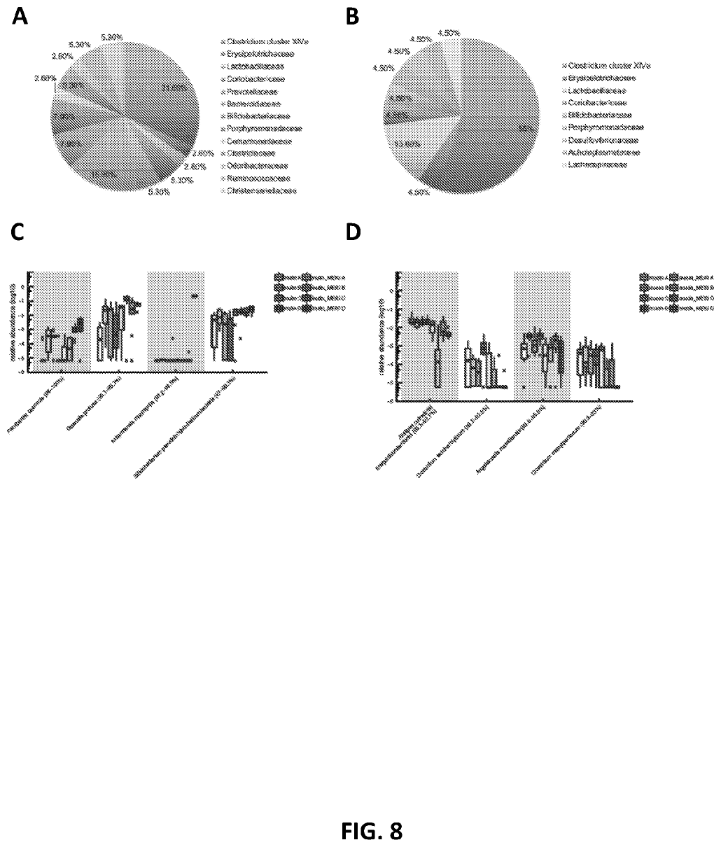

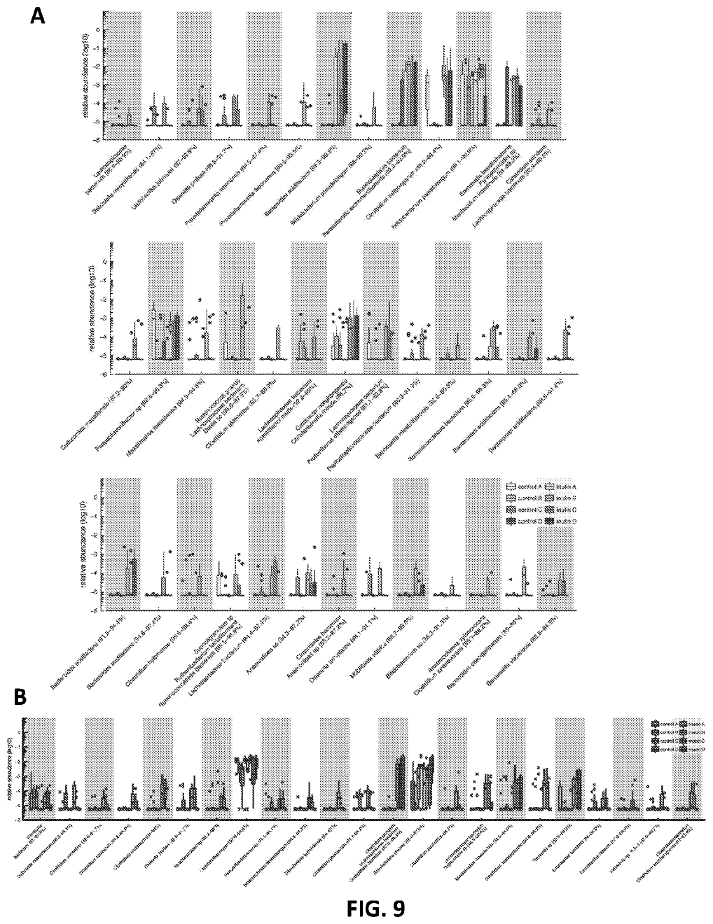

In the absence of MEK inhibitor (MEKi), inulin increased the relative abundance of 39 phylotype groups (FIG. 8A and FIG. 9A) that were negatively correlated with N-Ras mutant tumor size, whereas mucin enriched for 23 phylotype groups that were negatively correlated with tumor growth (FIG. 9B). Both inulin and mucin primarily increased the relative abundance of taxa mapped in or near Clostridium cluster XIVa (FIG. 8A-B). Among the phylotype groups induced by prebiotics, inulin specifically induced 6 phylotypes related to Bacteroides spp. (primarily B. acidifaciens and 3 phylotypes related to Barnesiella spp. and a notable increase in a phylotype group related to Parasutterella excrementihominis, not observed following mucin treatment. Inulin also increased the relative abundance of 3 phylotype groups related to Bifidobacterium, compared to only 1 phylotype group induced by mucin. The genomes of Bacteroides, Bifidobacterium and Barnesiella encode numerous glycosyl hydrolase activities. This catabolism supports a number of cross-feeding interactions with sugar fermenting bacteria, particularly members of the Clostridiales.

Mice treated with MEKi alone effectively controlled tumor growth and were associated with the enrichment of several phylotype groups, none of these phylotypes were negatively correlated with tumor size (TABLE 4).

Analysis of taxa following prebiotic combination with MEKi revealed that inulin induced 8 phylotype groups, enriched in the phylum Actinobacteria, including Bifidobacterium longum and two Olsenella spp. (TABLE 5). One phylotype group mapping distantly to Clostridium cellobioparum was negatively correlated with tumor size.

Mucin treatment resulted in the increased relative abundance of 56 phylotype groups featuring a broad diversity of taxa including Bacteroides, Parabacteroides, Olsenella and Clostridium (TABLE 6). Mucin uniquely increased the relative abundance of 5 Lactobacillus spp., all of which were positively correlated with tumor size, albeit none of these correlations were statistically significant. Without being bound by any particular theory, mucin likely increased the relative abundance of an excess of positively correlating phylotypes compared to negatively correlating taxa, resulting in a failure to control tumor growth in this experiment.

Twenty-one phylotype groups in inulin treated mice were altered in their relative abundance following MEKi injection and tumor inoculation, 5 of which were increased (TABLE 7), compared to mucin treated mice that displayed altered relative abundance of 15 phylotype groups, 6 of which were increased (TABLE 8). Analysis of the repertoire of phylotypes identified in insulin+MEKi treated mice, compared to those treated with inulin alone, revealed 4 groups that negatively correlated with tumor size based on their relative abundance at sacrifice (FIG. 8C). Akkermansia muciniphila was robustly enriched by inulin along with members of Actinobacteria, Bifidobacterium longum, Olsenella profusa and Parvibacter caecicola. While A. muciniphila has been demonstrated to possess anti-tumor properties, its induction in mice subjected to mucin in combination with MEKi implies that it may not be sufficient to control MaN-Ras tumor growth in this experiment. Without wishing to be bound by theory, interactions between taxa induced by inulin may be required for A. muciniphila's anti-tumor phenotype. In the mucin treated cohorts, 4 phylotype groups were identified that displayed negative correlations with tumor size in mice subjected to mucin+MEKi treatment, compared to mucin alone, which could be associated with the reduced relative abundance of these taxa (FIG. 8D).

TABLE 4 provides the abundance of phylotype groups enriched by MEKi administration (without mucin or inulin). Abundance C is at the time of MEKi injection. Abundance D is at the time of tumor collection. None of these phylotypes were negatively correlated with tumor size.

TABLE 4

| | Abundance | | p- |
|---|---|---|---|
| BLAST best hit/s (% identity) | C | D | value |
| Akkermansia muciniphila (100) | 0.001 | 15.879 | 0.00002 |
| Kineothrix alysoides (98) | 3.528 | 10.815 | 0.004 |
| Clostridium saccharolyticum (97) | | | |
| Muribaculum intestinale (87) | 0.014 | 0.534 | 0.048 |
| Ihubacter massiliensis (98) | 0.024 | 0.151 | 0.035 |
| E. timonensis (96) | | | |
| Barnesiella intestinihominis (85) | 0.028 | 0.145 | 0.047 |
| Pseudoflavonifractor phocaeensis (90) | 0.028 | 0.108 | 0.033 |

TABLE 5 provides the relative abundance of phylotype groups (osu) altered by MEKi combined with inulin. Abundances are provided for prior to inulin feeding and MEKi administration (A), and 14 days after inulin feeding and MEKi administration (B). Correlations are provided between the abundance of the phylotype group at time point B, and tumor size 20 days after tumor inoculation.

TABLE 5

| | | Abundance | | | Correlation | |
|---|---|---|---|---|---|---|
| | Other phylogenetic | A | B | | with tumor size | |
| BLAST best hit | descriptors | (log10) | (log 10) | p-value | rho | p-value |
| Anaerocolumna | Firmicutes, | −4.79 | −2.69 | 0.002 | −0.39 | 0.41 |
| jejuensis | Clostridia, | | | | | |
| | Clostridiales, | | | | | |
| | Clostridiaceae, | | | | | |
| | Lachnospiraceae, | | | | | |
| | Clostridium, | | | | | |
| | Anaerocolumna | | | | | |
| Bifidobacterium | Actinobacteria, | −4.80 | −4.30 | 0.007 | 0.22 | 0.62 |
| pseudolongum | Bifidobacteriales, | | | | | |
| | Bifidobacteriaceae, | | | | | |
| | Bifidobacterium | | | | | |
| Clostridium | Firmicutes, | −4.77 | −1.77 | 0.007 | −0.69 | 0.04 |
| cellobioparum | Clostridia, | | | | | |
| | Clostridiales, | | | | | |
| | Clostridiaceae, | | | | | |
| | Clostridium | | | | | |
| Natranaerovirga | Firmicutes, | −4.80 | −4.34 | 0.007 | 0.19 | 0.62 |
| pecinivora | Tissierellia, | | | | | |
| | Tissierellales, | | | | | |
| | Natranaerovirga | | | | | |
| Olsenella | Actinobacteria, | −4.80 | −4.08 | 0.010 | −0.22 | 0.62 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.80 | −4.62 | 0.010 | −0.05 | 0.86 |
| urininfantis | Coriobacteriia, | | | | | |
| | Coriobacteriales, | | | | | |
| | Atopobiaceae, | | | | | |
| | Olsenella | | | | | |
| Christensenella | Firmicutes, | −4.80 | −4.24 | 0.019 | −0.17 | 0.62 |
| minuta | Clostridia, | | | | | |
| | Clostridiales, | | | | | |
| | Christensenellaceae, | | | | | |
| | Catabacteriaceae, | | | | | |
| | Christensenella, | | | | | |
| | Catabacter | | | | | |
| Prevotellamassilia | Bacteroidetes, | −4.80 | −4.33 | 0.032 | −0.44 | 0.39 |
| timonensis | Bacteroidia, | | | | | |
| | Bacteroidales, | | | | | |
| | Prevotellaceae, | | | | | |
| | Prevotellamassilia | | | | | |

TABLE 6 provides the relative abundance of phylotype groups (osu) altered by MEKi combined with mucin. Abundances are provided for prior to mucin feeding and MEKi administration (A), and 14 days after mucin feeding and MEKi administration (B). Correlations are provided between the abundance of the phylotype group at time point B, and tumor size 20 days after tumor inoculation.

TABLE 6

| | | Abundance | | | Correlation | |
|---|---|---|---|---|---|---|
| | Other phylogenetic | A | B | | with tumor size | |
| BLAST best hit | descriptors | (log10) | (log 10) | p-value | rho | p-value |
| Alkalibacter | Firmicutes, | −4.87 | −4.82 | 0.012 | 0.40 | 0.76 |
| saccharofermentans | Clostridia, | | | | | |
| | Clostridiales, | | | | | |
| | Eubacteriaceae, | | | | | |
| | Alkalibacter | | | | | |
| Alloprevotella | Bacteroidetes, | −4.87 | −4.73 | 0.001 | 0.36 | 0.76 |
| rava | Bacteroidia, | | | | | |
| | Bacteroidales, | | | | | |
| | Prevotellaceae, | | | | | |
| | Alloprevotella | | | | | |
| Alloprevotella | Bacteroidetes, | −4.87 | −4.82 | 0.002 | 0.10 | 0.92 |
| rava | Bacteroidia, | | | | | |
| | Bacteroidales, | | | | | |
| | Prevotellaceae, | | | | | |
| | Alloprevotella, | | | | | |
| | Prevotellamassilia | | | | | |
| Aminiphila | Firmicutes, | −4.87 | −4.33 | 0.002 | 0.30 | 0.76 |
| butyica | Clostridia, | | | | | |
| | Clostridiales, | | | | | |
| | Clostridiaceae, | | | | | |
| | Aminiphila | | | | | |
| Bacteroides | Bacteroidetes, | −4.87 | −3.52 | 0.0005 | 0.29 | 0.76 |
| acidifaciens | Bacteroidia, | | | | | |
| | Bacteroidales, | | | | | |
| | Bacteroidaceae, | | | | | |
| | Bacteroides | | | | | |
| Bacteroides | Bacteroidetes, | −4.87 | −4.78 | 0.0005 | 0.21 | 0.91 |
| acidifaciens | Bacteroidia, | | | | | |
| | Bacteroidales, | | | | | |
| | Bacteroidaceae, | | | | | |
| | Bacteroides | | | | | |
| Barnesiella | Bacteroidetes, | −4.87 | −2.07 | 0.0005 | 0.31 | 0.76 |
| intestinihominis | Bacteroidia, | | | | | |
| | Bacteroidales, | | | | | |
| | Porphyromonadaceae, | | | | | |
| | Barnesiella, | | | | | |
| | Muribaculum, | | | | | |
| | Parabacteroides | | | | | |
| Barnesiella | Bacteroidetes, | −4.84 | −2.38 | 0.0005 | 0.31 | 0.76 |
| intestinihominis | Bacteroidia, | | | | | |
| | Bacteroidales, | | | | | |
| | Porphyromonadaceae, | | | | | |
| | Barnesiella | | | | | |
| Clostridium | Firmicutes, | −4.87 | −4.82 | 0.012 | −0.11 | 0.92 |
| aldenense | Clostridia, | | | | | |
| | Clostridiales, | | | | | |
| | Lachnospiraceae, | | | | | |
| | Clostridium | | | | | |
| Clostridium | Firmicutes, | −4.87 | −4.73 | 0.0005 | −0.22 | 0.91 |
| cocleatum | Clostridia, | | | | | |
| | Clostridiales, | | | | | |
| | Clostridiaceae, | | | | | |
| | Clostridium | | | | | |
| Clostridium | Firmicutes, | −4.87 | −4.78 | 0.002 | −0.08 | 0.92 |
| cocleatum | Clostridia, | | | | | |
| | Clostridiales, | | | | | |
| | Clostridiaceae, | | | | | |
| | Clostridium | | | | | |
| Clostridium | Firmicutes, | −4.87 | −4.47 | 0.001 | 0.01 | 0.99 |
| saccharogumia | Clostridia, | | | | | |
| | Clostridiales, | | | | | |
| | Clostridiaceae, | | | | | |
| | Clostridium, | | | | | |
| Clostridium | Firmicutes, | −4.87 | −4.78 | 0.027 | 0.60 | 0.24 |
| scindens | Clostridia, | | | | | |
| | Clostridiales, | | | | | |
| | Clostridiaceae, | | | | | |
| | Lachnospiraceae, | | | | | |
| | Clostridium | | | | | |
| Desulfovibrio | Proteobacteria, | −4.87 | −4.78 | 0.003 | 0.18 | 0.92 |
| fairfieldensis | Deltaproteobacteria, | | | | | |
| | Desulfovibrionales, | | | | | |
| | Desulfovibrionaceae, | | | | | |
| | Desulfovibrio | | | | | |
| Dubosiella | Firmicutes, | −4.87 | −4.82 | 0.005 | 0.32 | 0.76 |
| newyorkensis | Erysipelotrichia, | | | | | |
| | Erysipelotrichales, | | | | | |
| | Erysipelotrichaceae, | | | | | |
| | Dubosiella | | | | | |
| Enterobacter | Proteobacteria, | −4.87 | −4.78 | 0.002 | 0.12 | 0.92 |
| cloacae | Gammaproteobacteria, | | | | | |
| | Enterobacterales, | | | | | |
| | Enterobacteriaceae, | | | | | |
| | Enterobacter, | | | | | |
| | Klebsiella, | | | | | |
| | Escherichia, | | | | | |
| | Leclercia, | | | | | |
| | Yokenella | | | | | |
| Enterorhabdus | Actinobacteria, | −4.87 | −4.82 | 0.001 | 0.08 | 0.92 |
| muris | Coriobacteriia, | | | | | |
| | Eggerthellales, | | | | | |
| | Eggerthellaceae, | | | | | |
| | Enterorhabdus | | | | | |
| Eubacterium | Firmicutes, | −4.87 | −4.73 | 0.009 | 0.03 | 0.98 |
| dolichum | Clostridia, | | | | | |
| | Clostridiales, | | | | | |
| | Eubacteriaceae, | | | | | |
| | Eubacterium, | | | | | |
| | Absiella | | | | | |
| Flintibacter | Firmicutes | −4.87 | −4.82 | 0.005 | 0.01 | 0.99 |
| butyricus | | | | | | |
| Flintibacter | Firmicutes | −4.87 | −4.82 | 0.007 | 0.40 | 0.76 |
| butyricus | | | | | | |
| Intestinimonas | Firmicutes, | −4.87 | −4.46 | 0.001 | −0.05 | 0.96 |
| butyriciproducens | Clostridia, | | | | | |
| | Clostridiales, | | | | | |
| | Intestinimonas | | | | | |
| Intestinimonas | Firmicutes, | −3.31 | −2.70 | 0.009 | 0.42 | 0.76 |
| butyriciproducens | Clostridia, | | | | | |
| | Clostridiales, | | | | | |
| | Intestinimonas | | | | | |
| Intestinimonas | Firmicutes, | −4.87 | −4.82 | 0.001 | 0.11 | 0.92 |
| massiliensis | Clostridia, | | | | | |
| | Clostridiales, | | | | | |
| | Intestinimonas | | | | | |
| Lactobacillus | Firmicutes, | −4.87 | −4.82 | 0.009 | 0.07 | 0.92 |
| gasseri | Bacilli, | | | | | |
| | Lactobacillales, | | | | | |
| | Lactobacillaceae, | | | | | |
| | Lactobacillus | | | | | |
| Lactobacillus | Firmicutes, | −4.87 | −4.31 | 0.016 | 0.29 | 0.76 |
| hominis | Bacilli, | | | | | |
| | Lactobacillales, | | | | | |
| | Lactobacillaceae, | | | | | |
| | Lactobacillus | | | | | |
| Lactobacillus | Firmicutes, | −4.87 | −4.82 | 0.009 | 0.03 | 0.98 |
| johnsonii | Bacilli, | | | | | |
| | Lactobacillales, | | | | | |
| | Lactobacillaceae, | | | | | |
| | Lactobacillus | | | | | |
| Lactobacillus | Firmicutes, | −4.87 | −4.82 | 0.003 | 0.07 | 0.92 |
| reuteri | Bacilli, | | | | | |
| | Lactobacillales, | | | | | |
| | Lactobacillaceae, | | | | | |
| | Lactobacillus | | | | | |
| Lactobacillus | Firmicutes, | −4.87 | −4.82 | 0.009 | 0.25 | 0.83 |
| reuteri | Bacilli, | | | | | |
| | Lactobacillales, | | | | | |
| | Lactobacillaceae, | | | | | |
| | Lactobacillus | | | | | |
| Millionella | Millionella | −4.87 | −4.78 | 0.003 | 0.10 | 0.92 |
| massiliensis | | | | | | |
| Muribaculum | Bacteroidetes, | −4.87 | −4.82 | 0.007 | 0.30 | 0.76 |
| intestinale | Bacteroidia, | | | | | |
| | Bacteroidales, | | | | | |
| | Porphyromonadaceae, | | | | | |
| | Parabacteroides | | | | | |
| Muribaculum | Bacteroidetes, | −4.87 | −4.78 | 0.0005 | 0.08 | 0.92 |
| intestinale | Bacteroidia, | | | | | |
| | Bacteroidales | | | | | |
| Muribaculum | Bacteroidetes, | −4.86 | −3.93 | 0.001 | 0.07 | 0.92 |
| intestinale | Bacteroidia, | | | | | |
| | Bacteroidales, | | | | | |
| | Porphyromonadaceae, | | | | | |
| | Parabacteroides | | | | | |
| Muricomes | Firmicutes, | −4.83 | −3.09 | 0.021 | 0.42 | 0.76 |
| intestini | Clostridia, | | | | | |
| | Clostridiales, | | | | | |
| | Lachnospiraceae, | | | | | |
| | Muricomes | | | | | |
| Olsenella | Actinobacteria, | −4.83 | −2.47 | 0.0005 | −0.70 | 0.06 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.87 | −4.78 | 0.002 | 0.08 | 0.92 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.87 | −4.78 | 0.003 | 0.31 | 0.76 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.86 | −4.82 | 0.012 | 0.00 | 0.99 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.87 | −4.82 | 0.012 | −0.04 | 0.98 |
| profusa | Coriobacteriia, | | | | | |
| | Coriobacteriales, | | | | | |
| | Atopobiaceae, | | | | | |
| | Olsenella | | | | | |
| Olsenella | Actinobacteria, | −4.86 | −4.58 | 0.001 | 0.48 | 0.76 |
| urininfantis | Coriobacteriia, | | | | | |
| | Coriobacteriales, | | | | | |
| | Atopobiaceae, | | | | | |
| | Olsenella | | | | | |
| Parabacteroides | Bacteroidetes, | −4.87 | −4.78 | 0.003 | 0.08 | 0.92 |
| distasonis | Bacteroidia, | | | | | |
| | Bacteroidales, | | | | | |
| | Porphyromonadaceae, | | | | | |
| | Parabacteroides | | | | | |
| Parabacteroides | Bacteroidetes, | −4.87 | −4.78 | 0.007 | 0.08 | 0.92 |
| distasonis | Bacteroidia, | | | | | |
| | Bacteroidales, | | | | | |
| | Porphyromonadaceae, | | | | | |
| | Parabacteroides | | | | | |
| Parabacteroides | Bacteroidetes, | −4.87 | −4.78 | 0.0005 | 0.08 | 0.92 |
| merdae | Bacteroidia, | | | | | |
| | Bacteroidales, | | | | | |
| | Porphyromonadaceae, | | | | | |
| | Parabacteroides | | | | | |
| Parabacteroides | Bacteroidetes, | −4.87 | −4.78 | 0.0005 | 0.08 | 0.92 |
| merdae | Bacteroidia, | | | | | |
| | Bacteroidales, | | | | | |
| | Porphyromonadaceae, | | | | | |
| | Parabacteroides | | | | | |
| Parasutterella | Proteobacteria, | −4.87 | −4.73 | 0.0005 | 0.30 | 0.76 |
| excrementihominis | Betaproteobacteria, | | | | | |
| | Burkholderiales, | | | | | |
| | Sutterellaceae, | | | | | |
| | Parasutterella | | | | | |
| Prevotellamassilia | Bacteroidetes, | −4.83 | −1.07 | 0.0005 | −0.11 | 0.92 |
| timonensis | Bacteroidia, | | | | | |
| | Bacteroidales, | | | | | |
| | Prevotellaceae, | | | | | |
| | Prevotellamassilia | | | | | |
| Prevotellamassilia | Bacteroidetes, | −4.87 | −4.73 | 0.001 | 0.01 | 0.99 |
| timonensis | Bacteroidia, | | | | | |
| | Bacteroidales, | | | | | |
| | Prevotellaceae, | | | | | |
| | Prevotellamassilia | | | | | |
| Prevotellamassilia | Bacteroidetes, | −4.87 | −4.73 | 0.001 | 0.07 | 0.92 |
| timonensis | Bacteroidia, | | | | | |
| | Bacteroidales, | | | | | |
| | Prevotellaceae | | | | | |
| Prevotellamassilia | Bacteroidetes, | −4.87 | −4.73 | 0.016 | 0.27 | 0.78 |
| timonensis | Bacteroidia, | | | | | |
| | Bacteroidales, | | | | | |
| | Prevotellaceae, | | | | | |
| | Prevotellamassilia | | | | | |
| Provencibacterium | Firmicutes, | −4.87 | −4.03 | 0.005 | −0.09 | 0.92 |
| massiliense | Clostridia, | | | | | |
| | Clostridiales, | | | | | |
| | Ruminococcaceae, | | | | | |
| | Harryflintia, | | | | | |
| | Provencibacterium | | | | | |
| Pseudoflavonifractor | Firmicutes, | −4.86 | −4.82 | 0.042 | −0.24 | 0.88 |
| capillosus | Clostridia, | | | | | |
| | Clostridiales, | | | | | |
| | Pseudoflavonifractor | | | | | |
| Roseburia | Firmicutes, | −3.98 | −2.57 | 0.005 | 0.15 | 0.92 |
| intestinalis | Clostridia, | | | | | |
| | Clostridiales, | | | | | |
| | Lachnospiraceae, | | | | | |
| | Roseburia | | | | | |
| Roseburia | Firmicutes, | −4.87 | −4.14 | 0.034 | 0.18 | 0.92 |
| intestinalis | Clostridia, | | | | | |
| | Clostridiales, | | | | | |
| | Lachnospiraceae, | | | | | |
| | Roseburia | | | | | |
| Ruminococcus | Firmicutes, | −4.87 | −4.82 | 0.016 | −0.30 | 0.76 |
| gnavus | Clostridia, | | | | | |
| | Clostridiales, | | | | | |
| | Lachnospiraceae, | | | | | |
| | Ruminococcus | | | | | |
| Ruminococcus | Firmicutes, | −4.87 | −3.23 | 0.034 | 0.28 | 0.78 |
| lactaris | Clostridia, | | | | | |
| | Clostridiales, | | | | | |
| | Ruminococcaceae, | | | | | |
| | Lachnospiraceae, | | | | | |
| | Ruminococcus | | | | | |
| Sporanaerobacter | Firmicutes, | −4.87 | −4.82 | 0.002 | −0.18 | 0.92 |
| acetigenes | Tissierellia, | | | | | |
| | Tissierellales, | | | | | |
| | Sporanaerobacter | | | | | |
| Ureaplasma | Tenericutes, | −4.87 | −4.78 | 0.012 | 0.10 | 0.92 |
| urealyticum | Mollicutes, | | | | | |
| | Mycoplasmatales, | | | | | |
| | Mycoplasmataceae, | | | | | |
| | Ureaplasma | | | | | |

TABLE 7 compares the relative abundance of phylotype groups (OSU) of mice treated with inulin versus mice treated with inulin and MILK inhibitor (MEKi). Mice were inoculated with N-Ras mutant melanoma tumor cells, and microbial abundance determined after tumor growth and the indicated treatments.

TABLE 7

| | | Abundance (log10) | | |
|---|---|---|---|---|
| | Other phylogenetic | | Inulin + | p- |
| BLAST best hit | descriptors | Inulin | MEKi | value |
| Flintibacter | Firmicutes | −2.19 | −3.51 | 0.010 |
| butyricus | | | | |
| Olsenella | Actinobacteria, | −3.33 | −1.22 | 0.038 |
| profusa | Coriobacteriia, | | | |
| | Coriobacteriales, | | | |
| | Atopobiaceae, | | | |
| | Olsenella | | | |
| Akkermansia | Verrucomicrobia, | −4.53 | −0.68 | 0.004 |
| muciniphila | Verrucomicrobiae, | | | |
| | Verrucomicrobiales, | | | |
| | Akkermansiaceae, | | | |
| | Akkermansia | | | |
| Bifidobacterium | Actinobacteria, | −2.26 | −1.54 | 0.010 |
| pseudolongum | Actinobacteria, | | | |
| | Bifidobacteriales, | | | |
| | Bifidobacteriaceae, | | | |
| | Bifidobacterium | | | |
| Muribaculum | Bacteroidetes, | −1.06 | −1.89 | 0.027 |
| intestinale | Bacteroidia, | | | |
| | Bacteroidales, | | | |
| | Porphyromonadaceae, | | | |
| | Muribaculum | | | |
| Muribaculum | Bacteroidetes, | −4.51 | −1.63 | 0.004 |
| intestinale | Bacteroidia, | | | |
| | Bacteroidales, | | | |
| | Porphyromonadaceae, | | | |
| | Muribaculum | | | |
| Clostridium | Firmicutes, | −1.94 | −4.56 | 0.038 |
| saccharolyticum | Clostridia, | | | |
| | Clostridiales, | | | |
| | Clostridiaceae, | | | |
| | Lachnospiraceae, | | | |
| | Clostridium | | | |
| Oscillibacter | Firmicutes, | −2.22 | −3.15 | 0.035 |
| valericigenes | Clostridia, | | | |
| | Clostridiales, | | | |
| | Oscillospiraceae, | | | |
| | Oscillibacter | | | |
| Clostridium | Firmicutes, | −2.13 | −4.54 | 0.019 |
| indolis | Clostridia, | | | |
| | Clostridiales, | | | |
| | Clostridiaceae, | | | |
| | Clostridium | | | |
| Alistipes | Bacteroidetes, | −2.00 | −4.56 | 0.004 |
| senegalensis | Bacteroidia, | | | |
| | Bacteroidales, | | | |
| | Rikenellaceae, | | | |
| | Alistipes | | | |
| Parasutterella | Proteobacteria, | −1.97 | −1.51 | 0.004 |
| excrementihominis | Betaproteobacteria, | | | |
| | Burkholderiales, | | | |
| | Sutterellaceae, | | | |
| | Parasutterella | | | |
| Acetatifactor | Firmicutes, | −1.83 | −4.15 | 0.038 |
| muris | Clostridia, | | | |
| | Clostridiales, | | | |
| | Lachnospiraceae, | | | |
| | Acetatifactor, | | | |
| | Lachnoclostridium | | | |
| Clostridium | Firmicutes, | −3.09 | −4.55 | 0.019 |
| phoceensis | Clostridia, | | | |
| | Clostridiales, | | | |
| | Clostridiaceae, | | | |
| | Clostridium | | | |
| Anaerotaenia | Firmicutes, | −2.37 | −4.56 | 0.019 |
| torta | Clostridia, | | | |
| | Clostridiales, | | | |
| | Lachnospiraceae, | | | |
| | Lachnoclostridium | | | |
| Clostridium | Firmicutes, | −2.98 | −4.56 | 0.019 |
| saccharolyticum | Clostridia, | | | |
| | Clostridiales, | | | |
| | Clostridiaceae, | | | |
| | Clostridium | | | |
| Clostridium | Firmicutes, | −3.38 | −4.56 | 0.019 |
| xylanolyticum | Clostridia, | | | |
| | Clostridiales, | | | |
| | Clostridiaceae, | | | |
| | Clostridium | | | |
| Eisenbergiella | Firmicutes, | −2.67 | −4.56 | 0.035 |
| massiliensis | Clostridia, | | | |
| | Clostridiales, | | | |
| | Clostridiaceae, | | | |
| | Lachnospiraceae, | | | |
| | Clostridium, | | | |
| | Eisenbergiella | | | |
| Intestinimonas | Firmicutes, | −3.13 | −4.55 | 0.019 |
| massiliensis | Clostridia, | | | |
| | Clostridiales, | | | |
| | Intestinimonas, | | | |
| Dehalobacterium | Firmicutes, | −2.87 | −4.56 | 0.038 |
| formicoaceticum | Bacilli, | | | |
| | Bacillales, | | | |
| | Dehalobacterium | | | |
| Natranaerovirga | Firmicutes, | −3.34 | −4.56 | 0.038 |
| pectinivora | Clostridia, | | | |
| | Clostridiales, | | | |
| | Natranaerovirga | | | |
| Clostridium | Firmicutes, | −3.01 | −4.56 | 0.019 |
| xylanolyticum | Clostridia, | | | |
| | Clostridiales, | | | |
| | Clostridiaceae, | | | |
| | Clostridium | | | |

TABLE 8 compares the relative abundance of phylotype groups (OSU) of mice treated with mucin versus mice treated with mucin and MEK inhibitor (MEKi). Mice were inoculated with N-Ras mutant melanoma tumor cells, and microbial abundance determined after tumor growth and the indicated treatments.

TABLE 8

| | | Abundance (log10) | | |
|---|---|---|---|---|
| | Other phylogenetic | | Mucin + | p- |
| BLAST best hit | descriptors | Mucin | MEKi | value |
| Akkermansia | Verrucomicrobia, | −4.60 | −0.75 | 0.003 |
| muciniphila | Verrucomicrobiae, | | | |
| | Verrucomicrobiales, | | | |
| | Akkermansiaceae, | | | |
| | Akkermansia | | | |
| Parabacteroides | Bacteroidetes, | −0.91 | −1.56 | 0.005 |
| sp. YL27 | Bacteroidia, | | | |
| Muribaculum | Bacteroidales, | | | |
| intestinale | Porphyromonadaceae, | | | |
| YL27 | Muribaculum, | | | |
| | Parabacteroides | | | |
| Parabacteroides | Bacteroidetes, | −1.06 | −1.68 | 0.003 |
| sp. YL27 | Bacteroidia, | | | |
| | Bacteroidales, | | | |
| | Porphyromonadaceae, | | | |
| | Parabacteroides | | | |
| Parabacteroides | Bacteroidetes, | −4.60 | −2.24 | 0.009 |
| sp. YL27 | Bacteroidia, | | | |
| | Bacteroidales, | | | |
| | Porphyromonadaceae, | | | |
| | Parabacteroides | | | |
| Alistipes putredinis | Bacteroidetes, | −1.64 | −2.35 | 0.003 |
| DSM 17216, | Bacteroidia, | | | |
| Alistipes putredinis | Bacteroidales, | | | |
| JCM 16772, | Rikenellaceae, | | | |
| Alistipes onderdonkii | Alistipes | | | |
| WAL 8169 DSM 19147, | | | | |
| Alistipes sp. AL-1, | | | | |
| Alistipes finegoldii | | | | |
| 2789STDY5608890, | | | | |
| Alistipes finegoldii | | | | |
| 2789STDY5834947, | | | | |
| Alistipes onderdonkii | | | | |
| An90, | | | | |
| Alistipes onderdonkii | | | | |
| JCM 16771, | | | | |
| Alistipes onderdonkii | | | | |
| WAL 8169, | | | | |
| Alistipes sp. An66 | | | | |
| Clostridium | Firmicutes, | −1.76 | −3.75 | 0.047 |
| sp. KNHs209 | Clostridia, | | | |
| | Clostridiales, | | | |
| | Clostridiaceae, | | | |
| | Clostridium | | | |
| Clostridium | Firmicutes, | −3.15 | −4.61 | 0.036 |
| sp. M62/1, | Clostridia, | | | |
| Clostridium | Clostridiales, | | | |
| saccharolyticum | Clostridiaceae, | | | |
| An168 | Clostridium | | | |
| Clostridium | Firmicutes, | −2.21 | −4.20 | 0.014 |
| sp. M62/1, | Clostridia, | | | |
| Clostridium | Clostridiales, | | | |
| saccharolyticum | Clostridiaceae, | | | |
| An168 | Clostridium | | | |
| Burkholderiales | Proteobacteria, | −4.60 | −2.16 | 0.003 |
| bacterium | Betaproteobacteria, | | | |
| 1 1 47, | Burkholderiales, | | | |
| Parasutterella | Sutterellaceae, | | | |
| excrementihominis | Parasutterella | | | |
| YIT 11859 | | | | |
| Clostridium | Firmicutes, | −2.07 | −4.15 | 0.016 |
| phoceensis | Clostridia, | | | |
| GD3 | Clostridiales, | | | |
| | Clostridiaceae, | | | |
| | Clostridium | | | |
| Parabacteroides | Bacteroidetes, | −4.60 | −1.62 | 0.003 |
| sp. SN4 | Bacteroidia, | | | |
| | Bacteroidales, | | | |
| | Porphyromonadaceae, | | | |
| | Parabacteroides | | | |
| Clostridium | Firmicutes, | −2.71 | −4.61 | 0.036 |
| sp. ASF356 | Clostridia, | | | |
| | Clostridiales, | | | |
| | Clostridiaceae, | | | |
| | Clostridium | | | |
| Roseburia | Firmicutes, | −2.17 | −4.61 | 0.036 |
| sp. 831b | Clostridia, | | | |
| | Clostridiales, | | | |
| | Lachnospiraceae, | | | |
| | Roseburia | | | |
| Millionella | Millionella | −4.60 | −1.75 | 0.003 |
| massiliensis | | | | |
| Marseille-P3215 | | | | |
| Coprobacter | Bacteroidetes, | −4.60 | −2.17 | 0.003 |
| secundus | Bacteroidia, | | | |
| 177, | Bacteroidales, | | | |
| Gabonia | Porphyromonadaceae, | | | |
| massiliensis | Coprobacter, | | | |
| GM3 | Gabonia | | | |

### Example 10: Inulin Attenuates Colon Cancer Growth

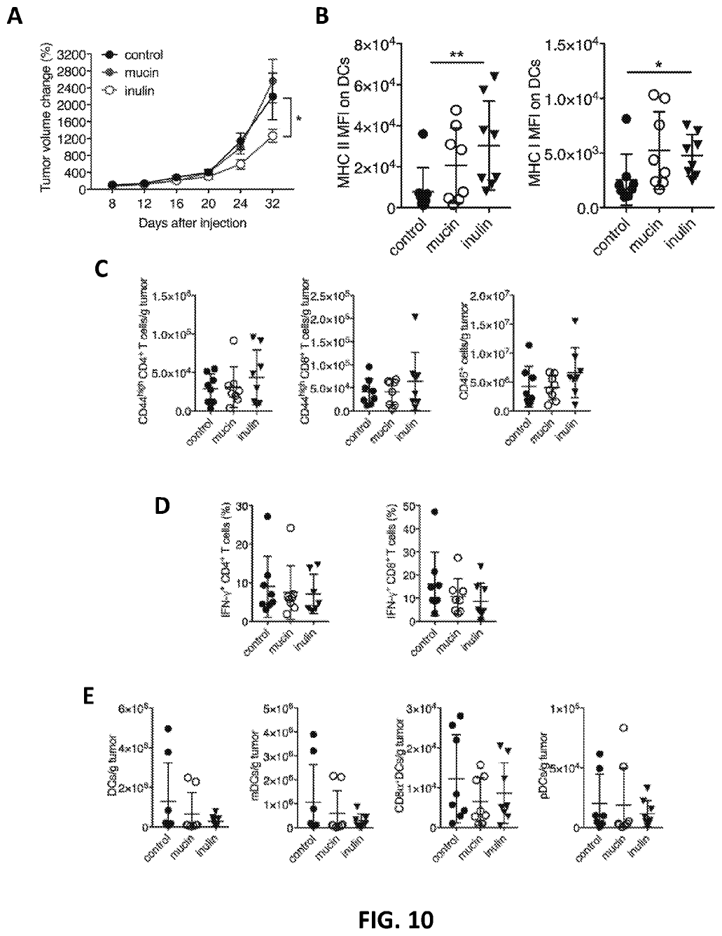

The effects of mucin and inulin on tumor growth were evaluated in a colon cancer model. C57BL/6 mice were fed with 3% mucin in drinking water, a diet enriched 15% inulin, or neither, for 14 days prior to tumor inoculation. MC-38 mouse colorectal cancer cells (1×10⁶) were inoculated, and diets were continued after inoculation. Inulin, but not mucin, administration for two weeks prior to tumor cell inoculation, attenuated the growth of colon cancer MC-38 tumors (FIG. 10A). Correspondingly, inulin-treated mice also exhibited enhanced anti-tumor immune responses, reflected in MHC class II and I expression on DCs (FIG. 10B). No differences in CD4+ or CD8+ T cells, CD45+ cells, cytokine production, or DCs and DC subsets were observed in tumors from mucin and inulin-treated mice (FIG. 10C-E).

### Example 11: Prebiotic-Induced Alterations in Microbiota Associated with Colon Cancer Control and Immunity

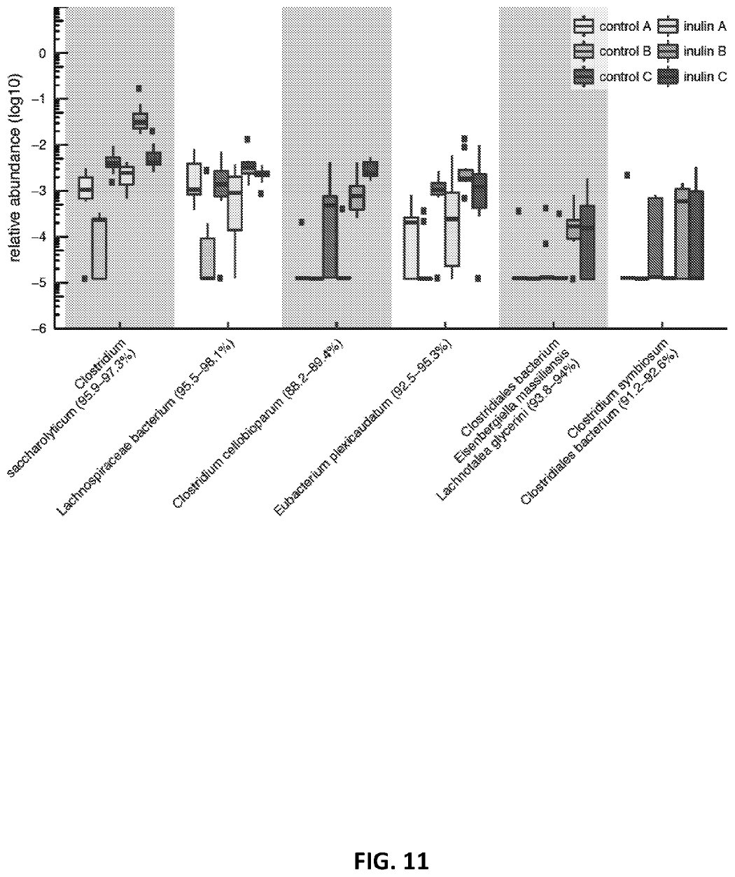

The effects of inulin on the microbiota were evaluated in a colon cancer model. C57BL/6 mice were fed with 3% mucin in drinking water, a diet enriched 15% inulin, or neither, for 14 days prior to tumor inoculation. MC-38 mouse colorectal cancer cells (1×10⁶) were inoculated, and diets were continued after inoculation. Analysis of fecal microbiota of mice treated with inulin and mucin indicated that both prebiotics increased the relative abundance of a similar number of phylotype groups (inulin induced 25 phylotype groups and mucin induced 21 phylotype groups). Of those, 7 phylotype groups were common to both prebiotics (TABLE 9 and TABLE 10). Notably, over 68% of the phylotype groups induced by inulin map to Clostridium cluster XIVa, compared with 33% induced by mucin. Additional analysis identified increased relative abundance of 6 inulin-specific phylotypes that were inversely correlated with tumor size (FIG. 11), whereas no phylotype groups induced by mucin were negatively correlated with tumor size.

TABLE 9 provides the abundance of phylotype groups (OSU groups) prior to inulin feeding (A), 14 days after inulin feeding (B), and 20 days post-tumor cell inoculation with inulin feeding (C). P-values were calculated using paired one-tail Wilcoxon rank sum test.

TABLE 9

| | Abundance | | | p-value | |
|---|---|---|---|---|---|
| BLAST best hit/s (% identity) | A | B | C | A vs B | B vs C |
| Dubosiella newyorkensis (99) | 0.032 | 1.521 | 1.246 | 0.030 | |
| Acetatifactor muris (97) | 0.144 | 0.338 | 0.168 | 0.006 | 0.021 |
| Parvibacter caecicola (100) | 0.008 | 0.114 | 0.005 | 2 × 10⁻⁹ | 5 × 10⁻¹⁰ |
| Adlercreutzia equolifaciens (96) | | | | | |
| Clostridium cocleatum (99) | 0.124 | 0.615 | 1.290 | 0.001 | |
| Olsenella profusa (95) | 0.060 | 2.229 | 2.023 | 0.0001 | |
| Kineothrix alysoides (96) | 0.006 | 0.146 | 0.158 | 0.010 | |
| Clostridium asparagiforme (95) | | | | | |
| Murimonas intestini (97) | 0.241 | 4.883 | 0.617 | 0.005 | 0.009 |
| Clostridium celerecrescens (96) XIVa | | | | | |
| Lachnoclostridium pacaense (96) | 0.127 | 0.323 | 0.165 | 0.007 | 0.038 |
| R. lactis (95) | | | | | |
| Eisenbergiella massileinsis (95) | | | | | |
| Bifidobacterium pseudolongum (99) | 0.414 | 2.337 | 1.700 | 0.001 | |
| Murimonas intestini (94) | 0.129 | 0.402 | 0.209 | 0.029 | |
| Roseburia hominis (94) | | | | | |
| Ruminococcus gnavus (94) | | | | | |
| Ruminococcus faecis (94) | | | | | |
| Clostridium cellobioparum (89) | 0.005 | 0.116 | 0.272 | 0.008 | 0.022 |
| Anaerotaenia torta (96) | 0.321 | 0.639 | | 0.002 | |
| C. xylanolyticum (95) | | | | | |
| Clostridium indolis (91) | 0.161 | 0.591 | 0.910 | 0.015 | |
| C. populeti (90) | | | | | |
| Bifidobacterium pseudolongum (99) | 0.383 | 1.381 | 0.964 | 0.009 | |
| Clostridium saccharolyticum (97) | 0.051 | 0.126 | 0.058 | 0.012 | |
| Eisenbergiella massiliensis (98) | 0.099 | 0.247 | 0.011 | 0.033 | 0.001 |
| C. asparigiforme (97) | | | | | |
| C. saccharolyticum (97) | | | | | |
| Flintibacter butyricus (94) | 0.015 | 0.033 | 0.015 | 0.024 | 0.011 |
| Pseudoflavonifractor capillosus (92) | | | | | |
| Pseudoflavonifractor phocaeensis (86) | 0.001 | 0.010 | 0.010 | 0.015 | |
| Intestinimonas butryiciproducens (86) | | | | | |
| Flintibacter butyricus (86) | | | | | |
| Desulfosporosinus fructosivorans (88) | 0.029 | 0.051 | 0.049 | 0.023 | |
| Ruminococcus lactaris (97) | 0.008 | 0.305 | 0.033 | 0.006 | 0.011 |
| Clostridium oroticum (97) | | | | | |
| Robinsoniella peoriensis (97) | 0.001 | 0.046 | 0.069 | 0.0001 | |
| Clostridium xylanolyticum (95) | 0.021 | 0.073 | 0.007 | 0.049 | 0.009 |
| Lachnoclostridium pacaense (95) | | | | | |
| Eisenbergiella massiliensis (94) | 0.004 | 0.021 | 0.036 | 0.046 | |
| Clostridium sufflavum (86) | 0.002 | 0.009 | | 0.038 | |
| C. cellulolyticum (86) | | | | | |
| Coprococcus comes (97) | 0.002 | 0.007 | | 0.047 | |
| Gracilibacter thermotolerans (89) | 0.002 | 0.036 | 0.009 | 0.007 | 0.040 |
| Christensenella massiliensis (88) | | | | | |
| Clostridium symbiosum (92) XIVa | 0.001 | 0.062 | 0.062 | 0.005 | |
| Clostridium xylanolyticum (96) | 0.001 | 0.117 | 0.021 | 0.040 | |
| Clostridium cellulolyticum (93) | 0.001 | 0.015 | 0.006 | 0.021 | |
| Clostridium asparagiforme (94) | 0.001 | 0.007 | 0.034 | | 0.034 |
| Clostridium oroticum (95) XIVa | 0.165 | 0.263 | 0.961 | | 0.020 |
| Pseudoflavonifractor phocaeensis (97) | 0.214 | 0.217 | 0.084 | | 0.001 |
| Bilophila wadsworthia (92) | 0.116 | 0.169 | 0.027 | | 0.0002 |
| Lachnoclostridium pacaense (97) | 0.001 | 0.007 | 0.296 | | 0.010 |
| C. indolis (97) | | | | | |
| C. saccharolyticum (97) | | | | | |
| Barnesiella intestinihominis (84) | 0.105 | 0.112 | 0.041 | | 0.023 |
| Clostridium populeti (95) | 0.120 | 0.128 | 0.325 | | 0.007 |
| Roseburia intestinalis (95) | 0.001 | 0.002 | 0.034 | | 0.045 |
| E. rectale | | | | | |
| Anaerotruncus rubiinfantis (89) | 0.023 | 0.065 | 0.013 | | 0.043 |
| Clostridium oroticum (99) | 0.042 | 0.062 | 0.001 | | 0.004 |
| Roseburia faecis (90) | 0.006 | 0.008 | 0.043 | | 0.013 |
| Faecalimonas umbilicata (97) | 0.004 | 0.009 | 0.042 | | 0.005 |
| Dorea formicigenerans (95) | | | | | |
| Anaerotruncus colihominis (98) | 0.055 | 0.057 | 0.003 | | 0.008 |
| Ruminococcus lactaris (91) | 0.001 | 0.003 | 0.044 | | 0.025 |
| Oscillibacter ruminantium (96) | 0.035 | 0.048 | 0.023 | | 0.005 |
| Actualibacter muris (100) | 0.023 | 0.035 | 0.010 | | 0.0001 |
| Neglecta timonensis (98) | | | | | |
| Clostridium saccharolyticum (96) | 0.021 | 0.226 | 0.769 | | 0.019 |

TABLE 10 provides the abundance of phylotype groups (OSU groups) prior to mucin feeding (A), 14 days after mucin feeding (B), and 20 days post-tumor cell inoculation with mucin feeding (C). P-values were calculated using paired one-tail Wilcoxon rank sum test.

TABLE 10

| | Abundance | | | p-value | |
|---|---|---|---|---|---|
| BLAST best hit/s (% identity) | A | B | C | A vs B | B vs C |
| Dubosiella newyorkensis (93) | 0.001 | 0.044 | 0.005 | 0.001 | 0.001 |
| Clostridium cocleatum (99) | 0.253 | 7.822 | 3.099 | 0.011 | |
| Olsenella profusa (92) | 0.001 | 0.118 | 0.068 | 0.023 | |
| Olsenella profusa (95) | 0.180 | 4.883 | 3.256 | 0.004 | |
| Muribaculum intesinale (92) | 6.008 | 17.058 | 11.857 | 4 × 10⁻⁷ | 0.007 |
| Murimonas intestini (97) | 0.104 | 0.300 | 0.462 | 0.009 | |
| Clostridium celerecrescens (96) XIVa | | | | | |
| Clostridium viride (96) | 0.013 | 0.062 | 0.113 | 0.001 | 0.007 |
| Intestinimonas butyriciproducens (96) | | | | | |
| Pseudoflavonifractor capillosus (95) | | | | | |
| Bifidobacterium pseudolongum (90) | 0.001 | 0.025 | 0.006 | 0.049 | |
| Clostridium xylanolyticum (98) | 0.014 | 0.088 | | 0.008 | |
| C. aerotolerans (97) | | | | | |
| Desulfovibrio desulfuricans (95) | 0.008 | 0.338 | 0.533 | 0.035 | |
| Christensenella timonensis (88) | 0.001 | 0.010 | 0.027 | 0.025 | 0.028 |
| Anaerotruncus colihominis (88) | | | | | |
| Anaerotaenia torta (96) | 0.168 | 0.421 | | 1 × 10⁻⁶ | |
| C. xylanolyticum (95) | | | | | |
| Roseburia faecis (95) | 0.013 | 0.124 | 0.555 | 0.009 | 0.011 |
| Ihubacter massiliensis (98) | 0.014 | 0.146 | | 0.002 | |
| E. timonensis (96) | | | | | |
| Eubacterium dolichum (92) | 0.045 | 0.291 | 0.462 | 0.00003 | |
| Eubacterium dolichum (93) | 0.011 | 0.106 | 0.279 | 0.025 | |
| Roseburia intestinalis (95) | 0.029 | 0.113 | 0.407 | 0.003 | 0.007 |
| Ruminococcus faecis (95) | | | | | |
| E. rectale (95) | | | | | |
| Clostridium cellulovorans (89) | 0.009 | 0.045 | 0.038 | 0.048 | |
| Christensenella massiliensis (88) | | | | | |
| Ruminococcus lactaris (97) | 0.004 | 0.062 | 0.060 | 0.0005 | |
| Clostridium oroticum (97) | | | | | |
| Harryflintia acetispora (95) | 0.017 | 0.040 | 0.172 | 0.043 | 0.006 |
| Anaerotruncus colihominis (93) | | | | | |
| Staphylococcus lentus (100) | 0.024 | 0.349 | 0.033 | 0.030 | 0.034 |
| Clostridium hylemonae (98) | 0.005 | 0.029 | 0.030 | 0.040 | |
| Muricomes intestini (98) | | | | | |
| Clostridium oroticum (98) XIVa | | | | | |
| Gracilibacter thermotolerans (89) | 0.001 | 0.006 | 0.020 | 0.036 | |
| Christensenella massiliensis (88) | | | | | |
| Staphylococcus saprophyticus (100) | 0.001 | 0.057 | 0.006 | 0.005 | 0.011 |
| Corynebacterium ammoniagenes (99) | 0.001 | 0.054 | 0.002 | 0.006 | 0.006 |
| Clostridium cellulolyticum (93) | 0.001 | 0.055 | 0.055 | 0.001 | |

### Example 12: Meta-Analysis of Anti-Tumor Microbiota

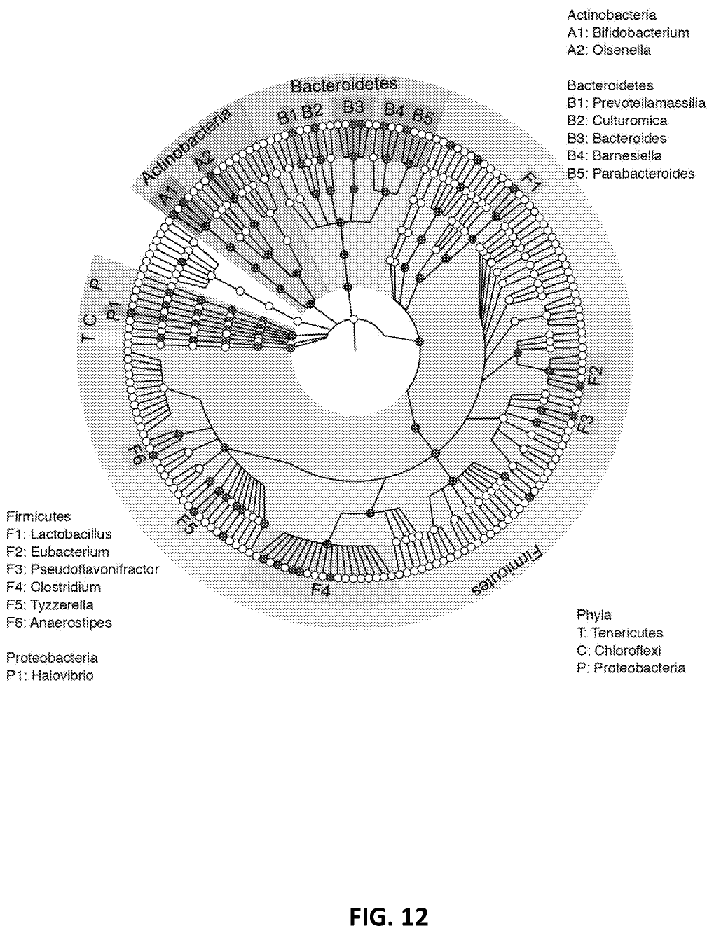

The taxa that negatively correlated with tumor size include multiple phylogenetic clades (FIG. 12). Among the distinct phylogenetic clades are bacterial strains encoding anti-tumor phenotypes, some of which have not previously been described. In this context, in addition to the previously reported Bifidobacterium taxa that has been implicated in anti-tumor responses, Olsenella spp. is identified herein (FIG. 12). Similarly, in addition to the previously reported Bacteroides, Barnesiella and Parabacteroides, the Prevotellamassilia, and Culturomica as additional taxa that inversely correlate with tumor size are identified herein. Six distinct species belonging to the Firmicutes were also associated with tumor growth inhibition featuring taxa mapping in or near Clostridium cluster XIVa (FIG. 12).

### Example 13: Mucin Induced Tumor Control is Dependent on Gut Microbiota

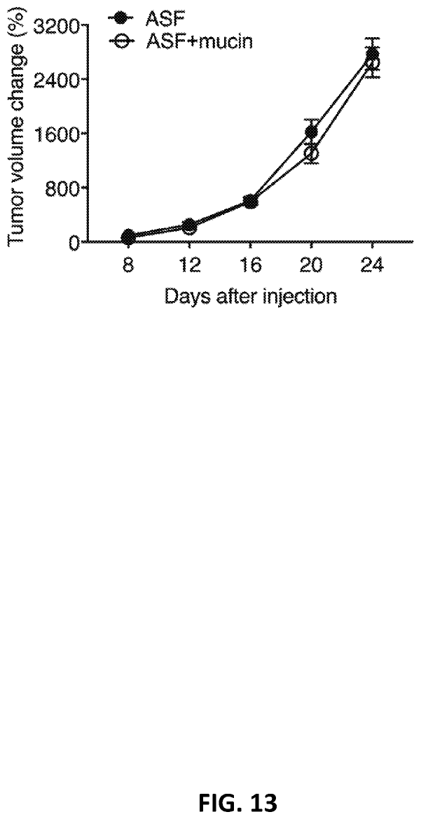

In order to test the requirement for gut microbiota in prebiotic-induced tumor control, experiments were conducted using mice with a defined, minimal microbiota. Germ free C3H/HeN mice were colonized with a minimal microbiota (ASF) to induce immune maturation for two weeks, followed by two weeks of mucin treatment of C3H/HeN mice at which time SW1 tumor cells were inoculated. Tumor size was monitored over the next 24 days. Mucin treated mice with a minimal microbiota failed to attenuate tumor growth (FIG. 13), in contrast to conventional mice in example 3, suggesting that tumor growth control seen in mucin-fed mice depends on specific gut microbiota.

### Example 14: Effect of Mucin and Inulin on the Activation of Dendritic Cells and T Cells

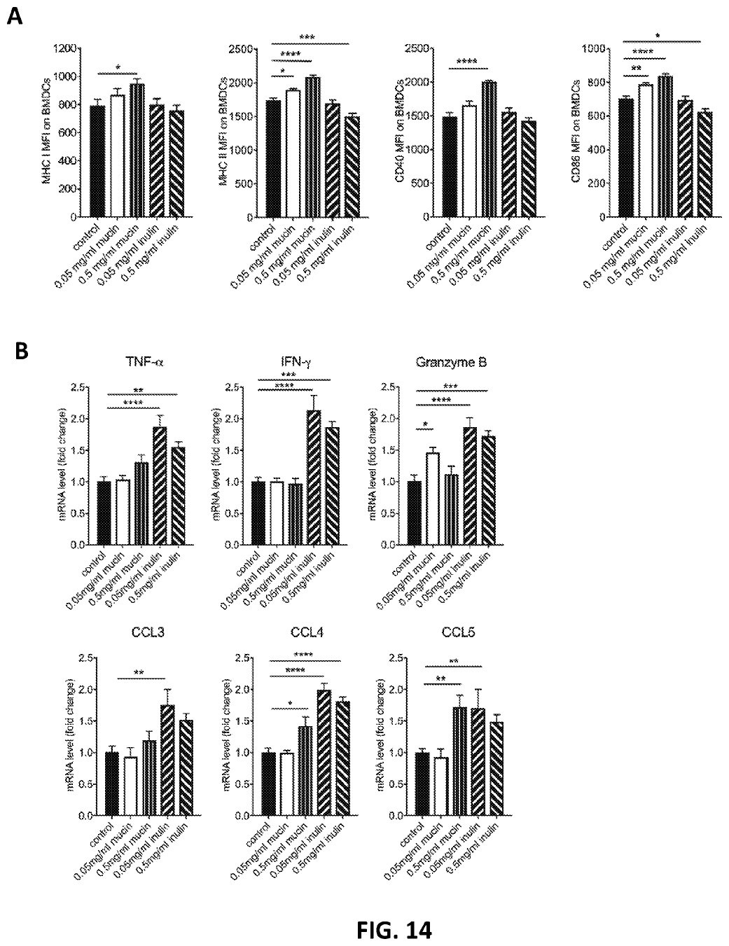

To further explore the potential direct effects of mucin or inulin on immune cells, murine bone marrow-derived dendritic cells (BMDCs) were cultured for 24 h with mucin or inulin at final concentration of 0.05 and 0.5 mg/ml. As shown in FIG. 14A, CD40 and CD80, markers for DC activation, as well as MHC I and MHC II on BMDCs were increased by mucin, but not inulin treatment. To evaluate effects on T cells, CD8+ T cells were isolated from spleen of normal mice and treated with different concentrations of mucin and inulin in vitro. Inulin treatment resulted in increased expression of multiple pro-inflammatory mediators, and mucin enhanced expression of Granzyme B, CCL4, and CCL5 in some conditions. (FIG. 14B). These results suggest that inulin and mucin can differentially affect expression of genes involved in dendritic cell antigen presentation, dendritic cell activation, and T cell effector functions.

### Example 15: Effect of Mucin and Inulin on Intestinal Epithelial Cells In Vivo

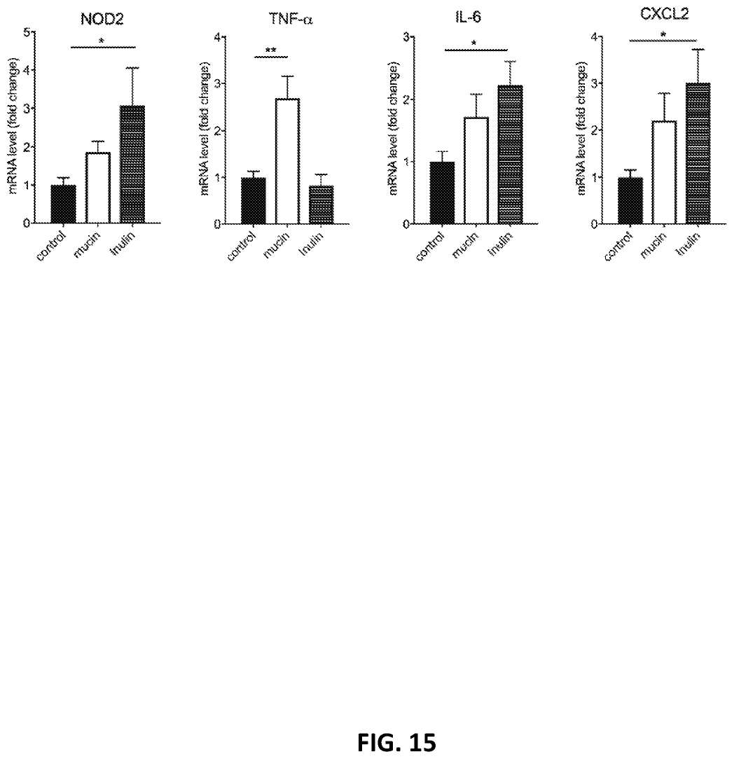

The effect of mucin and inulin on intestinal epithelial cells was evaluated in vivo. To this end, mucin (3% in drinking water) or inulin (15% in chow) were administered to naïve C57BL/6 mice for 2 weeks. Intestinal epithelial cells were isolated from small intestine and were assessed for the level of select cytokines and chemokines that are implicated in the activation of the immune response and anti-tumor immunity. Both prebiotics led to enhanced expression of select inflammatory chemokines and cytokines. While TNF-α mRNA level was elevated in mucin-treated mice, the levels of NOD2, IL-6 and CXCL2 mRNA were increased in inulin-treated mice (FIG. 15). These findings suggest that mucin and inulin can induce the transcription of cytokines and chemokines in intestinal epithelial cells, which have been implicated, for example, in the education of DC and activation of T cells. Without wishing to be bound by theory, prebiotic-induced alterations in the microbiota may elicit activation of the immune system and anti-tumor immunity via changes elicited in in intestinal epithelial cells.

### Example 16: Prebiotic Therapy Exhibits Comparable Efficacy as Anti-PD-1 Immune Checkpoint Therapy

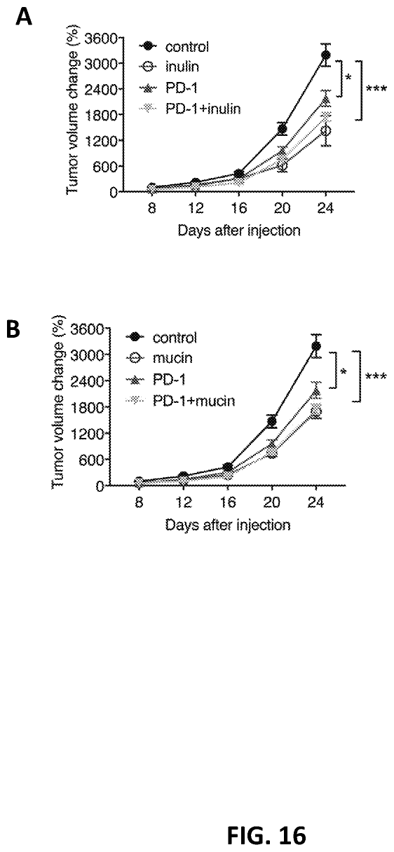

The ability of mucin and inulin to limit melanoma growth were compared to an anti-PD-1 antibody, one of the commonly used immune checkpoint therapies, particularly for melanoma. Notably, while administration of anti-PD-1 effectively limited the growth of YUMM1.5 BRAF mutant melanoma tumors in C57BL/6 mice, the effect of the prebiotics tested was just as potent (FIG. 16A-B). Combination of either prebiotic with PD-1 blockade did not result in greater attenuation of tumor growth (FIG. 16A-B). Without wishing to be bound by theory, mucin and inulin may elicit some changes that are comparable to those seen upon anti-PD1 therapy.

### Example 17: Tumor Growth Inhibition by Combination of Mucin and Inulin

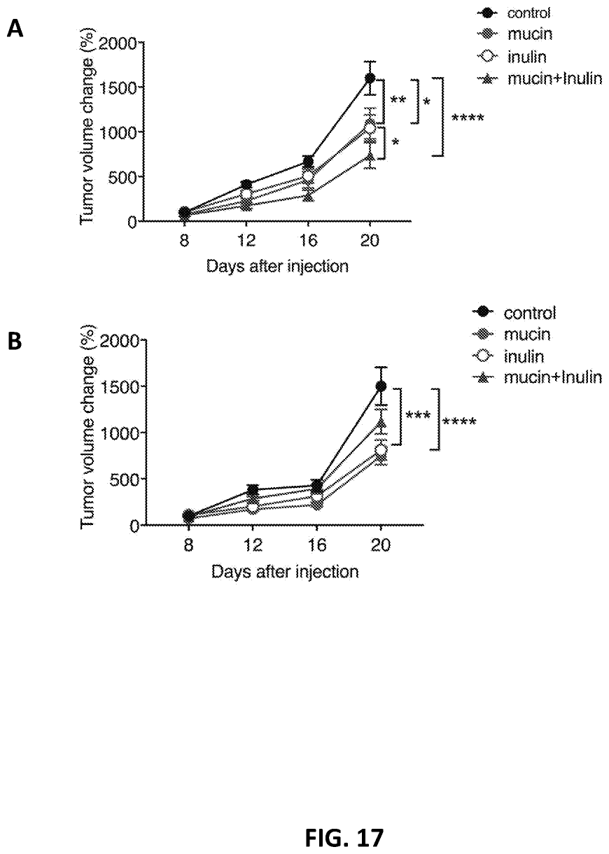

The effects of co-administering mucin and inulin were tested in two cancer models. Administration of mucin to C3H/HeOuJ mice that were inoculated with SW1 (syngeneic NRAS mutant melanoma) cells resulted in attenuated tumor growth, which was inhibited to a greater degree in the presence of inulin (FIG. 17A). However, no additive effect was observed when mucin and inulin were co-administered to C57BL/6 mice harboring BRAF melanoma tumors (FIG. 17B).

The examples and embodiments set forth herein are for illustrative purposes only and various modifications or changes suggested to persons skilled in the art are to be included within the spirit and purview of this application and scope of the appended claims.

What is claimed is:

1. A method of enhancing anti-cancer immunity comprising:
(a) administering to a subject a composition comprising mucin, wherein the subject has been identified as having a gut microbiome comprising one more microbial taxa that are members of a Clostridium cluster XIVa or an Actinobacteria phylum; and
(b) altering the gut microbiome in the subject,
wherein administration of the composition causes an enhanced anti-cancer immunity in the subject.

2. The method of claim 1, wherein the altering the gut microbiome comprises increasing an abundance of the one or more microbial taxa by at least 10%.

3. The method of claim 1, wherein the altering the gut microbiome comprises increasing an abundance of a microbial population by at least 10%.

4. The method of claim 3, wherein the microbial population is selected from the group consisting of: a microbial population that promotes inflammation, a microbial population that reduces inflammation, and a microbial population that is negatively correlated with tumor progression.

5. The method of claim 1, wherein the altering the gut microbiome comprises reducing an abundance of a microbial population by at least 10%.

6. The method of claim 5, wherein the microbial population is selected from the group consisting of: a microbial population that promotes inflammation, a microbial population that reduces inflammation, and a microbial population that is positively correlated with tumor progression.

7. The method of claim 1, wherein the altering the gut microbiome comprises increasing an abundance of a taxonomic unit by at least 10%.

8. The method of claim 7, wherein the taxonomic unit comprises a species selected from the group consisting of: a Clostriales species, a Bacteroides species, a Barnesiella species, a Parasutterella species, a Bifidobacterium species, an Olsenella species, a Parabacteroides species, a Dorea species, a Lachnospiraceae species, an Acetatifactor species, a Robinsoniella species, a Mobilitalea species, a Eubacterium species, an Eisenbergiella species, a Lachnotalea species, a Prevotellamassilia species, a Culturomica species, a Firmicutes species, a Pseudoflavonifractor species, a Tyzzerella species, an Anaerostipes species, a Proteobacteria species, a Halovibrio species, a Tenericutes species, and a Chlorflexi species.

9. The method of claim 1, wherein altering the gut microbiome comprises increasing a diversity of glycosyl hydrolases encoded by the gut microbiome by at least 10%.

10. The method of claim 1, wherein altering the gut microbiome comprises increasing a diversity of glycosyl hydrolases expressed by the gut microbiome by at least 10%.

11. The method of claim 1, wherein the method reduces tumor growth in the subject by at least 10%.

12. The method of claim 1, wherein the method reduces cancer progression in the subject.

13. The method of claim 12, wherein the cancer is a skin cancer.

14. The method of claim 12, wherein the cancer is a colorectal cancer.

15. The method of claim 1, further comprising administering to the subject an anti-cancer therapy.

16. The method of claim 15, wherein the anti-cancer therapy is selected from the group consisting of: radiotherapy, chemotherapy, immunotherapy, a chemical compound, a small molecule, a kinase inhibitor, a checkpoint inhibitor, and a cellular therapy.

17. The method of claim 15, wherein administering the anti-cancer therapy and the composition comprising mucin modifies the gut microbiome of the subject relative to administering only the composition comprising mucin.

18. The method of claim 15, wherein administering the anti-cancer therapy and the composition comprising mucin increases an abundance of a taxonomic unit by at least 10% relative to administering to the subject a composition comprising mucin.

19. The method of claim 18, wherein the taxonomic unit is selected from the group consisting of: an Akkermansia species, an Actinobacteria species, a Bifidobacterium species, an Olsenella species, and a Parvibacter species.

20. The method of claim 1, wherein the enhanced anti-cancer immunity is characterized by a stimulated anti-tumor immune response.

21. The method of claim 1, wherein the enhanced anti-cancer immunity is characterized by a stimulated pro-inflammatory immune response in a tumor microenvironment.

22. The method of claim 1, wherein the enhanced anti-cancer immunity comprises an increased tumor infiltration of at least 10% by cells selected from the group consisting of: CD4+ T cells, CD8+ T cells, CD45+ cells, dendritic cells, plasmacytoid dendritic cells, and CD8a+ dendritic cells.

23. The method of claim 1, wherein the enhanced anti-cancer immunity comprises an increased intra-tumoral expression of at least 10% of a gene selected from the group consisting of: an immune system gene, a cytokine gene, a chemokine gene, a gene involved in antigen presentation, a MHC-I gene, and a MHC-II gene.

24. The method of claim 1, wherein the method increases a concentration of a cytokine or chemokine in the subject's blood by at least 10%.

25. The method of claim 1, wherein the method decreases a concentration of a cytokine or chemokine in the subject's blood by at least 10%.

26. The method of claim 1, wherein the method increases expression of CD40, CD80, MHC-I, or MHC-II by dendritic cells in the subject by at least 10%.

27. The method of claim 1, wherein the method increases T cell activation in the subject by at least 10%.

28. The method of claim 1, wherein the method increases T cell expression of a cytokine, chemokine, or granzyme B in the subject by at least 10%.

29. The method of claim 1, wherein the method increases expression of an immune-related gene by intestinal epithelial cells in the subject by at least 10%.

30. The method of claim 1, wherein the method increases expression of a cytokine or chemokine by intestinal epithelial cells in the subject by at least 10%.

31. A method of enhancing anti-cancer immunity comprising:
(a) administering to a subject a composition comprising inulin, wherein the subject has been identified as having a gut microbiome comprising one more microbial taxa that are members of a Clostridium cluster XIVa or an Actinobacteria phylum; and
(b) altering the gut microbiome in the subject,
wherein administration of the composition causes an enhanced anti-cancer immunity in the subject.

32. The method of claim 31, wherein the altering the gut microbiome comprises increasing an abundance of the one or more microbial taxa by at least 10%.

33. The method of claim 31, wherein the altering the gut microbiome comprises increasing an abundance of a microbial population by at least 10%.

34. The method of claim 33, wherein the microbial population is selected from the group consisting of: a microbial population that promotes inflammation, a microbial population that reduces inflammation, and a microbial population that is negatively correlated with tumor progression.

35. The method of claim 31, wherein the altering the gut microbiome comprises reducing an abundance of a microbial population by at least 10%.

36. The method of claim 35, wherein the microbial population is selected from the group consisting of: a microbial population that promotes inflammation, a microbial population that reduces inflammation, and a microbial population that is positively correlated with tumor progression.

37. The method of claim 31, wherein the altering the gut microbiome comprises increasing an abundance of a taxonomic unit by at least 10%.

38. The method of claim 37, wherein the taxonomic unit comprises a species selected from the group consisting of: a Clostriales species, a Bacteroides species, a Barnesiella species, a Parasutterella species, a Bifidobacterium species, an Olsenella species, a Parabacteroides species, a Dorea species, a Lachnospiraceae species, an Acetatifactor species, a Robinsoniella species, a Mobilitalea species, a Eubacterium species, an Eisenbergiella species, a Lachnotalea species, a Prevotellamassilia species, a Culturomica species, a Firmicutes species, a Pseudoflavonifractor species, a Tyzzerella species, an Anaerostipes species, a Proteobacteria species, a Halovibrio species, a Tenericutes species, and a Chlorflexi species.

39. The method of claim 31, wherein altering the gut microbiome comprises increasing a diversity of glycosyl hydrolases encoded by the gut microbiome by at least 10%.

40. The method of claim 31, wherein altering the gut microbiome comprises increasing a diversity of glycosyl hydrolases expressed by the gut microbiome by at least 10%.

41. The method of claim 31, wherein the method reduces tumor growth in the subject by at least 10%.

42. The method of claim 31, wherein the method reduces cancer progression in the subject.

43. The method of claim 42, wherein the cancer is a skin cancer.

44. The method of claim 42, wherein the cancer is a colorectal cancer.

45. The method of claim 31, further comprising administering to the subject an anti-cancer therapy.

46. The method of claim 45, wherein the anti-cancer therapy is selected from the group consisting of: radiotherapy, chemotherapy, immunotherapy, a chemical compound, a small molecule, a kinase inhibitor, a checkpoint inhibitor, and a cellular therapy.

47. The method of claim 45, wherein administering the anti-cancer therapy and the composition comprising inulin modifies the gut microbiome of the subject relative to administering only the composition comprising inulin.

48. The method of claim 45, wherein administering the anti-cancer therapy and the composition comprising inulin increases an abundance of a taxonomic unit by at least 10% relative to administering to the subject a composition comprising inulin.

49. The method of claim 48, wherein the taxonomic unit is selected from the group consisting of: an Akkermansia species, an Actinobacteria species, a Bifidobacterium species, an Olsenella species, and a Parvibacter species.

50. The method of claim 31, wherein the enhanced anti-cancer immunity is characterized by a stimulated anti-tumor immune response.

51. The method of claim 31, wherein the enhanced anti-cancer immunity is characterized by a stimulated pro-inflammatory immune response in a tumor microenvironment.

52. The method of claim 31, wherein the enhanced anti-cancer immunity comprises an increased tumor infiltration of at least 10% by cells selected from the group consisting of: CD4+ T cells, CD8+ T cells, CD45+ cells, dendritic cells, plasmacytoid dendritic cells, and CD8a+ dendritic cells.

53. The method of claim 31, wherein the enhanced anti-cancer immunity comprises an increased intra-tumoral expression of at least 10% of a gene selected from the group consisting of: an immune system gene, a cytokine gene, a chemokine gene, a gene involved in antigen presentation, a MHC-I gene, and a MHC-II gene.

54. The method of claim 31, wherein the method increases a concentration of a cytokine or chemokine in the subject's blood by at least 10%.

55. The method of claim 31, wherein the method decreases a concentration of a cytokine or chemokine in the subject's blood by at least 10%.

56. The method of claim 31, wherein the method increases expression of CD40, CD80, MHC-I, or MHC-II by dendritic cells in the subject by at least 10%.

57. The method of claim 31, wherein the method increases T cell activation in the subject by at least 10%.

58. The method of claim 31, wherein the method increases T cell expression of a cytokine, chemokine, or granzyme B in the subject by at least 10%.

59. The method of claim 31, wherein the method increases expression of an immune-related gene by intestinal epithelial cells in the subject by at least 10%.

60. The method of claim 31, wherein the method increases expression of a cytokine or chemokine by intestinal epithelial cells in the subject by at least 10%.
